SEQUENCE LISTING (11 sequences)

SEQ ID NO: 1 - DNA, 746 bp, Homo sapiens
  CDS (56)...(442)
ccagcaggaggcacaggaaaactgcaagccgctctgttcctgggcctcggaagtgatgcctatggcgtcccctcaaaccctggtcctctatctgctggtcctggcagtcactgaagcctggggccaggaggcagtcatcccaggctgccacttgcaccccttcaatgtgacagtgcgaagtgaccgccaaggcacctgccagggctcccacgtggcacaggcctgtgtgggccactgtgagtccagcgccttcccttctcggtactctgtgctggtggccagtggttaccgacacaacatcacctccgtctctcagtgctgcaccatcagtggcctgaagaaggtcaaagtacagctgcagtgtgtggggagccggagggaggagctcgagatcttcacggccagggcctgccagtgtgacatgtgtcgcctctctcgctactagcccatcctctcccctccttcctcccctgggtcacagggcttgacattctggtgggggaaacctgtgttcaagattcaaaaactggaaggagctccagccctgatggttacttgctatggaatttttttaaataaggggagggttgttccagctttgatcctttgtaagattttgtgactgtcacctgagaagaggggagtttctgcttcttccctgcctctgcctggcccttctaaaccaatctttcatcattttacttccctctttgcccttacccctaaataaagcaagcagttcttga

SEQ ID NO: 2 - protein, 129 aa, Homo sapiens
MPMASPQTLVLYLLVLAVTEAWGQEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 3 - protein, 106 aa, Homo sapiens
QEAVIPGCHLHPFNVTVRSDRQGTCQGSHVAQACVGHCESSAFPSRYSVLVASGYRHNITSVSQCCTISGLKKVKVQLQCVGSRREELEIFTARACQCDMCRLSRY

SEQ ID NO: 4 - DNA, 390 bp, Homo sapiens
  CDS (1)...(390)
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatc

SEQ ID NO: 5 - protein, 130 aa, Homo sapiens
MKLAFLFLGPMALLLLAGYGCVLGASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 6 - protein, 106 aa, Homo sapiens
ASSGNLRTFVGCAVREFTFLAKKPGCRGLRITTDACWGRCETWEKPILEPPYIEAHHRVCTYNETKQVTVKLPNCAPGVDPFYTYPVAIRCDCGACSTATTECETI

SEQ ID NO: 7 - DNA, 5605 bp, Homo sapiens
atgaagctggcattcctcttccttggccccatggccctcctccttctggctggctatggctgtgtcctcggtgcctccagtgggaacctgcgcacctttgtgggctgtgccgtgagggagtttactttcctggccaagaagccaggctgcaggggccttcggatcaccacggatgcctgctggggtcgctgtgagacctgggaggtgagttgctaagttgtgcagatgacagtgtcttctaggccagcagcttgggtctgattcttaagagttcactttttaaatgatatgaggtagagctgggacatctgccctttcctgtggacttaaaaaaccaaaacaaaactatgattggcatcttccaaaagtgatttgaaaaacatgatgttgcccctctaacaaagcattgataaggttaagaatttggtttacattgtgtctatgtatctgggaatcatctctgggaggtcaagatgtactgttctacccgttttacagatgacatggagggattcaagggagagtggctgcaaagtcacgtagagcgtcagtgtaaagctgggaatcaatttgtggttcaagcttgtgacccaaactcctccctatgtttcctcattttggataaattagccagtttccaagaaagaggccctgagctgaagggtgagcgttggtcccagtgaagggtgagaccccttcactgcctcttctgcagcccttttcctcctcaagtctctgggagccctctggggttatcactgacggatccattaagttccttcatattcaattatacctggcctttttagagacatttaatttaaagtggagataacactctcaaacaaagttaaaatcctattgggctaagaggagctgtttgagtgatgaagaggaagagagctattcagcaccccagcagatcacattacgtagtgactgtgggctcttccccctgaggcctgcccacttggtaaccaatgaagtgctgtctctgatcttgtcactccctggcccaaaaaccttgaatgtccacacactactacagattcaataactaactttcaaggtgctcagcaatatggcgtctgcctgctttcctggagacagcacattttcttactctggccttggtaagtgactttcaaaggttttatcaaatagcccttatggatctcattttgttccttccctcatatcccttctccttcccatctgtcattatcatatttattcctgatgcctatctgcagtgccagctccctttctgggccttttttgacttgcaggtaagcccttgactatgctctacttttcgtcttacttcctcccccaccacacgcgtgatttaaattttttcaggacagaggttcattcttataaccttcacagcttttgtcaagatgtcgtgtatgaacaaggcattcaatacacatttgttggttgactgggatggacctccccctggagctgtagatcctccagcctaatggaaggccatttagaatcacacttgcactgtgagtggacactgccattgggaaaaatagccttctctttggggacccagagggtaacctgctcttgcttaggtacaattacggccctgtgaatggaattgggtcatagtgatgaaatctccaaattggatgaaactactctatcaaagtagttttcttttgcctcattcaggggcttgagccctactagcccaatgaaaatcgggttttgctaagtagactttgcctgtcaattggcagcaaattcacctggggcacttggcacctcctcctgttcagggactggcctggcagggcctctccctgttcgcatctagtgtctgggctatttgaagccctctctgtgccaaatcctcaaactcctgcttccgttcgattcagcccatcttctcttctttttaaaaactgatgaatgtctttaattggatcatggtcacccataggaggtcaggaactgtgctctcactggaaagatggaaacaccaaaaccgttaaagaacaagattctccctgatgttagccagctttcattcatgtcttgactgtgttatgaaaagggaggttacctatagaaaataaataaaagaatgagattcattttcccagcaatctgaaagtttctgcgctataaagcacttgattttttggtgggggggatcttaactgaaagcatgtctgaaaataaggatgttcatgatgacaggctggctggatttacatttgaaggttgttgaaaatagctattcctcataatctgggtatagagttgccagatttagcaaacaaacaaacagacaaacaaaataaaacaaaaccaatcccctccccacagaaacccaaactgaaataaaaccagaaaaccaggaagcccaggtaaatttgaatttaagataaataataaataaatttttagcataagtctgtctgtctcatacagtatttgggatgacttatactaaaaaattatgtatctgaaaatgaaattttatggggcgtttggtctgcctaggttcccagagtactaatggtaagaggacttaaagcaaatacgggaaggtaggagaaaacagttgaggacaaattcagctcttctggtctttgtcaaaggcaaggctggccgggcgtggtggctaacacctgtaatctcagcactttgggaggctgtggtgggtggataatgaggtcaggagttcgagaccagcctggccagtttttagtaaagaggtgagtaaaaccctgtctctactaaaaatacaaaaattagccgggcatggtggtatgcacctgtagtcccagctacttgggaggctgaggcagaagacttgcttgaacccaggaggtggaggttacagtgagccaagatcatgccactatactccagcctggcgacagagtgagactccatctcaaaaaaaaaaaaaaaaagaaaaaagaaaaaaaaaaggtaaggctgctattttcatgacattcatgcaagaacatcttgagttacatatgtatatatattcttttttgcctagaacaaagaagaaccaaaaagcaaaggtactgtcatttgaaagcttgttattatttacattactttcttataataattgcactaataagaacaatggattggctgggcgtggtggctcacgcctgtaatcccagcactttgggaggccgaggcaggcagatcacgaggtcaggaaatcgagaccatcctggctaacatggtgaaaccctgtctctactaaaaatacaaaaaatgagccaggcgtggtggtgggtgcctgtagtcccgggaggctgaggcaggagaatggcgtgaacccgggaggcggagattgcaatgagctgagattgcgccactgaactccagcctgggagacagcaagactccgtctcaaaaaaaaaaaaaaatggattgcattttttgaacatttactttgttctagacattgtgcattgcgtatatcatcttaccttatctctcaaacaatggtgggaggtagctattttgttttacagaggaggaaacttgagtcttcaggaagttaagtggattttccaaggtctccagcaagtggcagaacagggactcaagctccttagttctgactgcagggctcgagattttaactccagctaggtgctgatattttttctgatctgtgtgttctgtttatcaaaattgtctttgaacttaagatttataaaaggtgaaggaaggaaatgaatctttttgatgatcagaacagtgcacagagtattcgggaacctgtcttgtaatgttttctttcattgattcaatgacaaatagttattgaaactctcccagggtctgttttgggtacttgaggcacagtgggcaaaaatctctgtcctaaaagagcttactttctagagtgggaggaatatcacacgaatgaaaggtagactacgtcgtgtggtattgatcagtgctgtggtggaaaataaagcaagatgggggatgggaagtttctgggcatggagatggaatgttgcaattttaaataggatggtcaggaaatgcttccctgagagggtgacattctaacaaaaacccaaggttggtgaaagagtgaatcatacgggagaagaatgttccaggcagaaggaacagtaagtgcaaaggccctgagctggggctgttcctggtgggtcagaggagcaataaggagaccgccgtgagcctagtgaggaagtcagtgaggtgggaatggttgcaggcatttcagaaggtagagttgcagagaaggtgatgtaggtcttgaaggtgatcataaggtctttgatgtttgttctgagtgagatgggaaatcactggggctttgggcagaggagtaacatgatctgacttaggtttaaacaggatcactcagggccgctgtgttgcaaatagattgtagggagtaaaaatggaagaggggagaccagttagaaggtatttgcaatgactaagatgattcatttgctgactatgcatggagcacttgctgtgtgctatggtctctcctgggagcttagaatatggtcttgagtgaaatcagcttcttgctttcaggagtttgttttctactgggagacgacagagcaacaagtaaatcaacgaataacaagttaatttctgatagtgataaatgatactaaaaaactgaaacaagatcatatgttctaatgaattctctgtttctatctatggggacagaaacccattctggaacccccctatattgaagcccatcatcgagtctgtacctacaacgagaccaaacaggtgactgtcaagctgcccaactgtgccccgggagtcgaccccttctacacctatcccgtggccatccgctgtgactgcggagcctgctccactgccaccacggagtgtgagaccatctgaggccgctagctgctctctgcagacccacctgtgtgagcagcacatgcagttatacttcctggatgcaagactgtttaatttcgaccacacccatggaggaggttacctgtcgccccttaggtccagctcaggcaaaaggcccaaatgcagcctacttatgctaaaagttcaaaacaatattcgtgccttcaccaaaataatttctccagctcacatacctgcaaattaatttttctttgccttgagtcttggaacataatttgtgtatcacaatcctcccccaatttggacttataatatgctaatgatttaaacacatgggatgtaattaggatatggggctggaaagtctttaaattctcatgttctatttaacctctgatctccaaccggatttatgattaaagggctagaaatgaacaaaacccatgtactagtcttccttaccccagaggaattccagctgcaagcttctttagggaaaatgctcccttccccttttaactgagcaattatctacacaagaaataagactgctcagatatacaaagagagtagcttcaatgaaaagatgtttggatttggataattcttttccctagcaaaattcgctagctcccttaagagtcttaataaagaggctacgttgggattaaaagaaaaaaaaacagaaataaaatat

SEQ ID NO: 8 - DNA, 22 bp, Homo sapiens
tcagaagaaaatcagaggaatc

SEQ ID NO: 9 - DNA, 23 bp, Homo sapiens
gggacgttcagtagcggttgtag

SEQ ID NO: 10 - DNA, 20 bp, Homo sapiens
ctgcccatggacaccgagac

SEQ ID NO: 11 - DNA, 23 bp, Homo sapiens
ccgtttgcatatactcttctgag

(19) United States
(12) Patent Application Publication
(10) Pub. No.: US 2007/0111945 A1
(43) Pub. Date: May 17, 2007

(54) USE OF CORTICOTROPH-DERIVED GLYCOPROTEIN HORMONE TO TREAT INFLAMMATION AND POTENTIATE GLUCOCORTICOID ACTION

(75) Inventors: James D. Kelly, Mercer Island, WA (US); Philippa J. Webster, Seattle, WA (US); Stacey R. Dillon, Seattle, WA (US); Shannon L. O'Hogan, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(73) Assignee: ZymoGenetics, Inc.

(21) Appl. No.: 11/562,767

(22) Filed: Nov. 22, 2006

Related U.S. Application Data
(62) Division of application No. 10/459,000, filed on Jun. 10, 2003.
(60) Provisional application No. 60/387,322, filed on Jun. 10, 2002.

(51) Int. Cl. A61K 38/24

(52) U.S. Cl. 514/12

(57) ABSTRACT

The use of corticotroph-derived glycoprotein hormone (CGH) to potentiate glucocorticoid action and to reduce glucocorticoid-induced adrenocortical suppression is described. CGH can be co-administered with glucocorticoids to enable a lower dosage of glucocorticoids to be used and to prevent or reduce glucocorticoid-induced side-effects. The invention additionally provides methods for the use of CGH as a replacement for glucocorticoids, or for treatment of a broad range of inflammatory states. Further provided are methods for purification of recombinant CGH.

### REFERENCE TO RELATED APPLICATIONS

This application is a divisional of U.S. application Ser. No. 10/459,000, filed Jun. 10, 2003, which claims the benefit of U.S. Provisional Application Ser. No. 60/387,322, filed Jun. 10, 2002, both of which are herein incorporated by reference.

### BACKGROUND OF THE INVENTION

Inflammation normally is a localized, protective response to trauma or microbial invasion that destroys, dilutes, or walls-off the injurious agent and the injured tissue. Diseases characterized by inflammation are significant causes of morbidity and mortality in humans. While inflammation commonly occurs as a defensive response to invasion of the host by foreign material, it is also triggered by a response to mechanical trauma, toxins, and neoplasia. Excessive inflammation caused by abnormal recognition of host tissue as foreign, or prolongation of the inflammatory process, may lead to inflammatory diseases such as diabetes, asthma, atherosclerosis, cataracts, reperfusion injury, cancer, post-infectious syndromes such as in infectious meningitis, and rheumatic fever and rheumatic diseases such as systemic lupus erythematosus and rheumatoid arthritis. Thus, there is a need to produce agents that inhibit inflammation in many such diseases.

Glucocorticoids are used therapeutically as replacement therapy for individuals having adrenal insufficiencies, due to pathologies in the hypothalamus, anterior pituitary or the adrenal cortex. The glucocorticoids are also used for the treatment of a diverse number of non-endocrine diseases. Except in patients receiving replacement or substitution therapy, glucocorticoids are neither specific nor curative: they provide symptomatic relief by virtue of their anti-inflammatory and immunosuppressive properties. Glucocorticoids are used to treat rheumatic disorders such as rheumatoid arthritis, systemic lupus erythematosus, and a variety of vasculitic disorders such as polyarteritis nodosa, Wegener's granulomatosis and giant cell arteritis. In non-inflammatory degenerative joint diseases (e.g., osteoarthritis) or in a variety of regional pain syndromes (e.g., tendonitis or bursitis), glucocorticoids may be administered by local injection for the treatment of episodic disease flare-up.

Glucocorticoids are used to treat renal diseases, allergic disease including hay fever, serum sickness, urticaria, contact dermatitis, drug reactions, bee stings, allergic rhinitis and angioneurotic edema.

Glucocorticoids are also used to treat bronchial asthma, chronic obstructive pulmonary disease, chronic bronchitis and emphysema. Typically agents such as methylprednisolone or prednisone are used. Also inhaled glucocorticoids such as beclomethasone dipropionate, triamcinolone acetonide, flunisolide or budesonide can be used.

Glucocorticoids are used to treat a wide range of skin diseases including psoriasis, dermatitis, hidradenitis suppurativa, scabies, pityriasis rosea, lichen planus, and pityriasis rubra pilaris. Other inflammatory conditions in which glucocorticoids have been useful are toxic epidermal necrolysis, erythema multiforme, and sunburn.

Inflammatory bowel disease, ulcerative colitis and Crohn's disease can be treated with glucocorticoids. Glucocorticoids are also useful to treat chronic active hepatitis, alcoholic liver disease and severe hepatic disease. Glucocorticoids are used in the chemotherapy of acute lymphocytic leukemia and lymphomas because of their antilymphocytic effects. Glucocorticoids are also useful in the treatment of sarcoidosis, thrombocytopenia, autoimmune destruction of erythrocytes, organ transplantation, and in stroke and spinal cord injury.

However, as useful as glucocorticoids are, they do have severe side-effects. Two categories of toxic effects result from the therapeutic use of glucocorticoids: those resulting from withdrawal of glucocorticoid therapy and those resulting from continued use of supraphysiological doses. The most severe complication of the termination of glucocorticoid treatment is acute adrenal insufficiency, which results from too rapid a withdrawal of glucocorticoids after prolonged therapy, in which the hypothalamus/pituitary/adrenal (HPA) axis has been suppressed. Besides the consequences that result from the suppression of the HPA system, there are a number of other complications that result from prolonged glucocorticoid therapy, including fluid and electrolyte abnormalities, hypertension, hyperglycemia, increased susceptibility to infection, cataracts, growth arrest, fat redistribution, striae, ecchymosis, acne, hirsutism, and thymic atrophy.

Thus, there is a need to provide novel therapies to treat inflammation, including those administered in conjunction with glucocorticoid therapies that lessen the side-effects of glucocorticoid treatment.

The present invention provides such polypeptides for these and other uses that should be apparent to those skilled in the art from the teachings herein.

### DESCRIPTION OF THE INVENTION

Within an aspect of the invention, there is provided a method for treating inflammation, comprising administering a therapeutically sufficient amount of a CGH polypeptide to a mammal, wherein administration of the polypeptide results in a clinically significant improvement in the inflammatory condition of the mammal. Within an embodiment, the CGH polypeptide forms a heterodimer, comprising the amino acid sequence as shown in SEQ ID NO:3, and the amino acid sequence as shown in SEQ ID NO:6. Within another embodiment, the clinically significant improvement in the inflammatory condition is selected from the group consisting of: a decrease or inhibition in pain; a decrease or inhibition in swelling; a decrease or inhibition in redness; a decrease or inhibition in heat; and a decrease or inhibition in loss of function.

Within another aspect of the invention, there is provided a method for treating inflammation, comprising administering a therapeutically sufficient amount of a CGH polypeptide to a mammal, wherein the inflammation is acute or chronic. Within an embodiment, the inflammation or inflammatory condition is associated with an autoimmune disease.

Within another aspect of the invention, there is provided a method for treating inflammation, comprising administering a therapeutically sufficient amount of a CGH polypeptide to a mammal, wherein the inflammation is associated with a rheumatic disorder. The rheumatic disorder can be rheumatoid arthritis, system lupus erythematosus, a vasculitic disorder, or another rheumatic disorder.

Within another aspect of the invention, there is provided a method for treating inflammation, comprising administering a therapeutically sufficient amount of a CGH polypeptide to a mammal, wherein the inflammation is associated with an allergic response.

Within another aspect of the invention, there is provided a method for treating inflammation, comprising administering a therapeutically sufficient amount of a CGH polypeptide to a mammal, wherein the inflammation is located in the respiratory tract.

Within an embodiment, the inflammation is located in the lung, or sinus. Within another embodiment, the inflammation is associated with asthma, chronic obstructive pulmonary disease, chronic bronchitis, or emphysema.

Within another aspect of the invention, there is provided a method for treating inflammation, comprising administering a therapeutically sufficient amount of a CGH polypeptide to a mammal, wherein the inflammation is located on the epidermis. Within an embodiment, the inflammation is associated with psoriasis, or dermatitis.

Within another aspect of the invention, there is provided a method for treating inflammation, comprising administering a therapeutically sufficient amount of a CGH polypeptide to a mammal, wherein the inflammation is located in the gastrointestinal tract.

Within an embodiment, the inflammation is associated with Inflammatory Bowel disease, ulcerative colitis, Crohn's disease, or inflammation associated diarrhea.

Within another aspect of the invention, there is provided a method for treating inflammation, comprising administering a therapeutically sufficient amount of a CGH polypeptide to a mammal, wherein the inflammation is associated with Graft versus Host Disease. Within an embodiment, the inflammation is associated with single-organ or multi-organ failure.

Within another aspect of the invention, there is provided a method for treating inflammation, comprising administering a therapeutically sufficient amount of a CGH polypeptide to a mammal, wherein the inflammation is associated with sepsis.

Within another aspect of the invention, there is provided a method for treating inflammation, comprising administering a therapeutically sufficient amount of a CGH polypeptide to a mammal, wherein the inflammation is located in the liver. Within an embodiment, the inflammation is associated with chronic active hepatitis, alcoholic liver disease, or non-alcoholic fatty liver disease.

Within another aspect of the invention, there is provided a method for treating inflammation, comprising administering a therapeutically sufficient amount of a CGH polypeptide to a mammal, wherein the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.

Within another aspect of the invention, there is provided a method for treating inflammation, comprising administering a therapeutically sufficient amount of a CGH polypeptide to a mammal, wherein treatment with the CGH polypeptide is used as an alternative to glucocorticoid treatment.

Within another aspect of the invention, there is provided a method for treating inflammation, comprising administering a therapeutically sufficient amount of a CGH polypeptide to a mammal, wherein treatment with the CGH polypeptide prevents or reduces a glucocorticoid-induced adverse side-effect. Within an embodiment, the glucocorticoid-induced adverse side-effect is selected from the group consisting of: adrenocortical suppression, osteoporosis, bone necrosis, steroid-induced cataracts, steroid-induced obesity, corticosteroid-induced psychosis, gastrointestinal hemorrhage, thymic atrophy, and benign intracranial hypertension.

Within another aspect of the invention, there is provided a method for reducing inflammation, comprising administering a therapeutically sufficient amount of a CGH polypeptide to a mammal, wherein administration of the polypeptide results in a clinically significant improvement in the inflammatory condition of the mammal. Within an embodiment, the CGH polypeptide forms a heterodimer, comprising the amino acid sequence as shown in SEQ ID NO:3, and the amino acid sequence as shown in SEQ ID NO:6. Within another embodiment, the clinically significant improvement in the inflammatory condition is selected from the group consisting of: a decrease or inhibition in pain; a decrease or inhibition in swelling; a decrease or inhibition in redness; a decrease or inhibition in heat; and a decrease or inhibition in loss of function.

Within another aspect of the invention, there is provided a method for reducing inflammation, comprising administering a therapeutically sufficient amount of a CGH polypeptide to a mammal, wherein the inflammation is acute or chronic. Within an embodiment, the inflammation or inflammatory condition is associated with an autoimmune disease.

Within another aspect of the invention, there is provided a method for reducing inflammation, comprising administering a therapeutically sufficient amount of a CGH polypeptide to a mammal, wherein the inflammation is associated with a rheumatic disorder. The rheumatic disorder can be rheumatoid arthritis, system lupus erythematosus, a vasculitic disorder, or another rheumatic disorder.

Within another aspect of the invention, there is provided a method for reducing inflammation, comprising administering a therapeutically sufficient amount of a CGH polypeptide to a mammal, wherein the inflammation is associated with an allergic response.

Within another aspect of the invention, there is provided a method for reducing inflammation, comprising administering a therapeutically sufficient amount of a CGH polypeptide to a mammal, wherein the inflammation is located in the respiratory tract. Within an embodiment, the inflammation is located in the lung, or sinus. Within another embodiment, the inflammation is associated with asthma, chronic obstructive pulmonary disease, chronic bronchitis, or emphysema.

Within another aspect of the invention, there is provided a method for reducing inflammation, comprising administering a therapeutically sufficient amount of a CGH polypeptide to a mammal, wherein the inflammation is located on the epidermis. Within an embodiment, the inflammation is associated with psoriasis, or dermatitis.

Within another aspect of the invention, there is provided a method for reducing inflammation, comprising administering a therapeutically sufficient amount of a CGH polypeptide to a mammal, wherein the inflammation is located in the gastrointestinal tract. Within an embodiment, the inflammation is associated with Inflammatory Bowel disease, ulcerative colitis, Crohn's disease, or inflammation associated diarrhea.

Within another aspect of the invention, there is provided a method for reducing inflammation, comprising administering a therapeutically sufficient amount of a CGH polypeptide to a mammal, wherein the inflammation is associated with Graft versus Host Disease. Within an embodiment, the inflammation is associated with single-organ or multi-organ failure.

Within another aspect of the invention, there is provided a method for reducing inflammation, comprising administering a therapeutically sufficient amount of a CGH polypeptide to a mammal, wherein the inflammation is associated with sepsis.

Within another aspect of the invention, there is provided a method for reducing inflammation, comprising administering a therapeutically sufficient amount of a CGH polypeptide to a mammal, wherein the inflammation is located in the liver. Within an embodiment, the inflammation is associated with chronic active hepatitis, alcoholic liver disease, or non-alcoholic fatty liver disease.

Within another aspect of the invention, there is provided a method for reducing inflammation, comprising administering a therapeutically sufficient amount of a CGH polypeptide to a mammal, wherein the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.

Within another aspect of the invention, there is provided a method for reducing inflammation, comprising administering a therapeutically sufficient amount of a CGH polypeptide to a mammal, wherein treatment with the CGH polypeptide is used as an alternative to glucocorticoid treatment.

Within another aspect of the invention, there is provided a method for reducing inflammation, comprising administering a therapeutically sufficient amount of a CGH polypeptide to a mammal, wherein treatment with the CGH polypeptide prevents or reduces a glucocorticoid-induced adverse side-effect. Within an embodiment, the glucocorticoid-induced adverse side-effect is selected from the group consisting of: adrenocortical suppression, osteoporosis, bone necrosis, steroid-induced cataracts, steroid-induced obesity, corticosteroid-induced psychosis, gastrointestinal hemorrhage, thymic atrophy, and benign intracranial hypertension.

Within an aspect of the invention, there is provided a method for treating inflammation, comprising administering a therapeutically sufficient amount of a CGH polypeptide to a mammal in conjunction with one or more glucocorticoids, wherein administration of the polypeptide results in a clinically significant improvement in the inflammatory condition of the mammal. Within an embodiment, the CGH polypeptide forms a heterodimer, comprising the amino acid sequence as shown in SEQ ID NO:3, and the amino acid sequence as shown in SEQ ID NO:6. Within another embodiment, the clinically significant improvement in the inflammatory condition is selected from the group consisting of: a decrease or inhibition in pain; a decrease or inhibition in swelling; a decrease or inhibition in redness; a decrease or inhibition in heat; and a decrease or inhibition in loss of function.

Within another aspect of the invention, there is provided a method for treating inflammation, comprising administering a therapeutically sufficient amount of a CGH polypeptide to a mammal in conjunction with one or more glucocorticoids, wherein the inflammation is acute or chronic. Within an embodiment, the inflammation or inflammatory condition is associated with an autoimmune disease.

Within another aspect of the invention, there is provided a method for treating inflammation, comprising administering a therapeutically sufficient amount of a CGH polypeptide to a mammal in conjunction with one or more glucocorticoids, wherein the inflammation is associated with a rheumatic disorder. The rheumatic disorder can be rheumatoid arthritis, system lupus erythematosus, a vasculitic disorder, or another rheumatic disorder.

Within another aspect of the invention, there is provided a method for treating inflammation, comprising administering a therapeutically sufficient amount of a CGH polypeptide to a mammal in conjunction with one or more glucocorticoids, wherein the inflammation is associated with an allergic response.

Within another aspect of the invention, there is provided a method for treating inflammation, comprising administering a therapeutically sufficient amount of a CGH polypeptide to a mammal in conjunction with one or more glucocorticoids, wherein the inflammation is located in the respiratory tract. Within an embodiment, the inflammation is located in the lung, or sinus. Within another embodiment, the inflammation is associated with asthma, chronic obstructive pulmonary disease, chronic bronchitis, or emphysema.

Within another aspect of the invention, there is provided a method for treating inflammation, comprising administering a therapeutically sufficient amount of a CGH polypeptide to a mammal in conjunction with one or more glucocorticoids, wherein the inflammation is located on the epidermis. Within an embodiment, the inflammation is associated with psoriasis, or dermatitis.

Within another aspect of the invention, there is provided a method for treating inflammation, comprising administering a therapeutically sufficient amount of a CGH polypeptide to a mammal in conjunction with one or more glucocorticoids, wherein the inflammation is located in the gastrointestinal tract. Within an embodiment, the inflammation is associated with Inflammatory Bowel disease, ulcerative colitis, Crohn's disease, or inflammation associated diarrhea.

Within another aspect of the invention, there is provided a method for treating inflammation, comprising administering a therapeutically sufficient amount of a CGH polypeptide to a mammal in conjunction with one or more glucocorticoids, wherein the inflammation is associated with Graft versus Host Disease. Within an embodiment, the inflammation is associated with single-organ or multi-organ failure.

Within another aspect of the invention, there is provided a method for treating inflammation, comprising administering a therapeutically sufficient amount of a CGH polypeptide to a mammal in conjunction with one or more glucocorticoids, wherein the inflammation is associated with sepsis.

Within another aspect of the invention, there is provided a method for treating inflammation, comprising administering a therapeutically sufficient amount of a CGH polypeptide to a mammal in conjunction with one or more glucocorticoids, wherein the inflammation is located in the liver. Within an embodiment, the inflammation is associated with chronic active hepatitis, alcoholic liver disease, or non-alcoholic fatty liver disease.

Within another aspect of the invention, there is provided a method for treating inflammation, comprising administering a therapeutically sufficient amount of a CGH polypeptide to a mammal in conjunction with one or more glucocorticoids, wherein the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.

Within another aspect of the invention, there is provided a method for treating inflammation, comprising administering a therapeutically sufficient amount of a CGH polypeptide to a mammal in conjunction with one or more glucocorticoids, wherein treatment with the CGH polypeptide prevents or reduces a glucocorticoid-induced adverse side-effect. Within an embodiment, the glucocorticoid-induced adverse side-effect is selected from the group consisting of: adrenocortical suppression, osteoporosis, bone necrosis, steroid-induced cataracts, steroid-induced obesity, corticosteroid-induced psychosis, gastrointestinal hemorrhage, thymic atrophy, and benign intracranial hypertension.

Within another aspect of the invention, there is provided a method for reducing inflammation, comprising administering a therapeutically sufficient amount of a CGH polypeptide to a mammal in conjunction with one or more glucocorticoids, wherein administration of the polypeptide results in a clinically significant improvement in the inflammatory condition of the mammal. Within an embodiment, the CGH polypeptide forms a heterodimer, comprising the amino acid sequence as shown in SEQ ID NO:3, and the amino acid sequence as shown in SEQ ID NO:6. Within another embodiment, the clinically significant improvement in the inflammatory condition is selected from the group consisting of: a decrease or inhibition in pain; a decrease or inhibition in swelling; a decrease or inhibition in redness; a decrease or inhibition in heat; and a decrease or inhibition in loss of function.

Within another aspect, the invention provides a method for treating inflammation, comprising administering the CGH polypeptide and the glucocorticoid concurrently. Within another aspect, the invention provides a method for treating inflammation, comprising administering the CGH polypeptide and the glucocorticoid sequentially. Within another aspect, the invention provides a method for treating inflammation, comprising administering the CGH polypeptide and the glucocorticoid, wherein the glucocorticoid is short-acting. Within an embodiment, the glucocorticoid is cortisone, prednisone, prednisolone, or methylprednisolone. Within another aspect, the invention provides a method for treating inflammation, comprising administering the CGH polypeptide and the glucocorticoid, wherein the glucocorticoid is intermediate acting. Within an embodiment, the glucocorticoid is triamcinolone. Within another aspect, the invention provides a method for treating inflammation, comprising administering the CGH polypeptide and the glucocorticoid, wherein the glucocortocoid is long-acting. Within another embodiment, the glucocorticoid is dexamethasone or beta methasone.

Within another aspect of the invention, there is provided a method for treating inflammation, comprising administering a therapeutically sufficient amount of a CGH polypeptide to a mammal in conjunction with one or more glucocorticoids, wherein glucocorticoid is selected from the group consisting of alclometasone dipropionate, amcinonide, beclomethasone dipropionate, betamethasone, betamethasone benzoate, betamethasone dipropionate, betamethasone sodium, betamethasone valerate, clobetasol propionate, clocortolone pivalate, hydrocortisone, hydrocortisone acetate, hydrocortisone butyrate, hydrocortisone cypionate, hydrocortisone sodium phosphate, hydrocortisone sodium succinate, hydrocortisone valerate, cortisone acetate, desonide, desoximetasone, dexamethasone, dexamethasone acetate, dexamethasone sodium, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium, mometasone furoate, paramethasone acetate, prednislone, prednislone acetate, prednislone sodium phosphate, prednisolone tebutate, prednisone, triamcinolone, triamcinolone acetonide, triamcinolone diacetate and triamcinolone hexacetonide. Within an embodiment, the glucocorticoid is administered as a derivative of alclometasone dipropionate, amcinonide, beclomethasone dipropionate, betamethasone, betamethasone benzoate, betamethasone dipropionate, betamethasone sodium, betamethasone valerate, clobetasol propionate, clocortolone pivalate, hydrocortisone, hydrocortisone acetate, hydrocortisone butyrate, hydrocortisone cypionate, hydrocortisone sodium phosphate, hydrocortisone sodium succinate, hydrocortisone valerate, cortisone acetate, desonide, desoximetasone, dexamethasone, dexamethasone acetate, dexamethasone sodium, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium, mometasone furoate, paramethasone acetate, prednislone, prednislone acetate, prednislone sodium phosphate, prednisolone tebutate, prednisone, triamcinolone, triamcinolone acetonide, triamcinolone diacetate or triamcinolone hexacetonide.

Within an aspect of the invention, there is provided a method for treating inflammation, comprising administering a therapeutically sufficient amount of a CGH polypeptide to a mammal, wherein administration of the polypeptide results in a decrease of a pro-inflammatory indicator. Within an embodiment, the pro-inflammatory indicator is measured by serum levels or pro-inflammatory cytokines. Within an embodiment, the pro-inflammatory cytokine is TNFα. Within another embodiment the pro-inflammatory indicator is measured by a decrease in inflammation associated neutrophil infiltration

Within another aspect, the invention provides a method for forming a peptide-receptor complex comprising, providing an immobilized receptor; and contacting the receptor with a peptide, wherein the peptide comprises the amino acid sequence as shown in SEQ ID NO:3 and the receptor is TSHR; whereby the receptor binds the peptide.

Within another aspect, the invention provides a method for purifying CGH contained within a cell culture supernatant liquid comprising:

applying the CGH-containing supernatant liquid to a chromatography column containing a cation exchange resin under conditions wherein the CGH binds to said cation exchange resin;

eluting the CGH from the cation exchange resin and capturing a CGH-containing pool;

applying the CGH-containing pool to a chromatography column containing a hydrophobic interaction resin under conditions wherein the CGH binds to said hydrophobic interaction resin;

eluting the CGH from the hydrophobic interaction resin and capturing a CGH containing pool;

applying the CGH-containing pool to a size-exclusion column and eluting the CGH from the size-exclusion resin and capturing the CGH in a CGH-containing pool.

These and other aspects of the invention will become evident upon reference to the following detailed description of the invention.

### DETAILED DESCRIPTION OF THE INVENTION

The present invention provides a novel therapy to treat diseases for which a glucocorticoid is administered. The present invention thus comprises a method of administering corticotroph-derived glycoprotein hormone alone or in conjunction with a glucocorticoid to an individual having a disease for which a glucocorticoid is administered.

The teachings of all of the references cited herein are incorporated in their entirety herein by reference.

Corticotroph-derived glycoprotein hormone (CGH) is a heterodimeric protein hormone released from corticotroph cells in the anterior pituitary. CGH is disclosed in International Patent Application No. PCT/US01/09999, publication no. WO 01/73034. It is comprised of an alpha subunit, glycoprotein hormone alpha2 (GPHA2), and a beta subunit, glycoprotein hormone beta 5 (GPHB5). GPHA2 was previously called Zsig51 (International Patent Application No. PCT/US99/03104, publication no. WO 99/41377 published Aug. 19, 1999; U.S. Pat. No. 6,573,363). SEQ ID NO: 1 is the human cDNA sequence that encodes the full-length polypeptide GPHA2, and SEQ ID NO:2 is the full-length polypeptide sequence of human GPHA2. SEQ ID NO:3 is the mature GPHA2 polypeptide sequence without the signal sequence. SEQ ID NO: 4 is the human cDNA sequence that encodes the full-length GPHB5 polypeptide. SEQ ID NO: 5 is the full-length GPHB5 polypeptide. SEQ ID NO: 6 is the mature GPHB5 polypeptide without the signal sequence. SEQ ID NO: 7 is the human genomic DNA sequence that encodes the full-length GPHB5 polypeptide. The present invention also includes CGH polypeptides, and polynucleotides, that are substantially homologous to those of the SEQ ID NOs: 1, 2, 3, 4, 5, 6, and 7.

CGH is released from the same cells that produce adrenocorticotrophic hormone (ACTH), the primary regulator of the adrenal cortex. ACTH stimulates synthesis and release of glucocorticoid (GC) and androgenic hormones from the adrenal cortex. CGH targets the adrenal cortex and tissues that respond to glucocorticoids, such as cells of the immune system. The action of CGH and ACTH together as corticoptrophic hormones represents a novel paradigm for the regulation of the steroid-mediated stress response. The use of CGH to potentiate the actions of glucocorticoids and relieve adrenocortical suppression is described.

Examples of glucocorticoid steroids that can be administered in conjunction with CGH include alclometasone dipropionate, amcinonide, beclomethasone dipropionate, betamethasone, betamethasone benzoate, betamethasone dipropionate, betamethasone sodium, betamethasone vale rate, clobetasol propionate, clocortolone pivalate, hydrocortisone, hydrocortisone acetate, hydrocortisone butyrate, hydrocortisone cypionate, hydrocortisone sodium phosphate, hydrocortisone sodium succinate, hydrocortisone valerate, cortisone acetate, desonide, desoximetasone, dexamethasone, dexamethasone acetate, dexamethasone sodium, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium, mometasone furoate, paramethasone acetate, prednisolone, prednislone acetate, prednisolone sodium phosphate, prednisolone tebutate, prednisone, triamcinolone, triamcinolone acetonide, triamcinolone diacetate and triamcinolone hexacetonide.

### CGH Structure and Localization

GPHA2 is 25% identical in amino acid sequence to the common alpha subunit of the known glycoprotein hormones, and is predicted to have similar structural motifs. GPHB5 is approximately 30% identical in sequence to the beta subunits of human chorionic gonadotropin, thyroid-stimulating hormone, follicle-stimulating hormone, and luteinizing hormone, and is also predicted to be structurally conserved. GPHA2 does not dimerize with any of the other glycoprotein hormone beta subunits, nor does GPHB5 dimerize with the common alpha subunit. As shown in Example 5, when co-expressed in the same cell, GPHA2 and GPHB5 form a non-covalent heterodimer, CGH, which contains two N-linked glycosylations on the GPHA2 subunit and one N-linked glycosylation on the GPHB5 subunit.

CGH is produced and stored in the corticotroph cells in the anterior pituitary. See Example 1. Corticotrophs are one of six distinct cell types in the anterior pituitary, as characterized by distribution, histology, structure, and hormone content. See Molitch, in Endocrinology and Metabolism (Felig and Frohman, eds), pp. 111-171, McGraw-Hill, (2001) and Asa et al., in Endocrinology (DeGroot and Jameson, eds) Vol. 1, pp. 167-182, W.B. Saunders, (2001).

Corticotrophs, or ACTH-producing cells, constitute approximately 20% of anterior pituitary cells and are basophilic and periodic acid Schiff (PAS)-positive. Corticotrophs can also be identified by immunostaining with specific antisera to ACTH. CGH and ACTH are contained within the large number of secretory granules in these cells, and are released upon stimulation of the cells by corticotrophin-releasing hormone (CRH). In addition, AVP (antidiuretic hormone) acts in an additive fashion with CRH on corticotrophs.

### Regulation of ACTH and CGH Release from the Pituitary

Although a variety of substances in the blood may influence ACTH and CGH secretion, CRH exerts the primary control of the release of these hormones from the anterior pituitary. CRH is released from neurons in the paraventricular nucleus (PVN) of the hypothalamus in a pulsatile and phasic pattern. Stimulation of cholinergic transmission as well as excitatory amino acid neurotransmitters are considered to be important activators of CRH release. Nitric oxide is also intimately involved in the regulation of CRH release, and nitric oxide synthase (NOS) is co-localized with CRH in neurons in the PVN. Cytokines may stimulate CRH release by activation of NOS. Opioids inhibit CRH release from the PVN.

The circadian rhythm of CRH release is generated by variation of pulse amplitude. Peak levels are reached at approximately 6 a.m., decline during the day to 4 p.m., then further decline to a nadir between 11 p.m. and 3 a.m. See Snyder, in Endocrinology and Metabolism (Felig and Frohman, eds), pp. 173-216, McGraw-Hill, (2001). ACTH levels respond in parallel to CRH; GC levels also respond in parallel, but are delayed by approximately 30 minutes.

CRH stimulates ACTH secretion from the pituitary in a sustained, biphasic manner. Initially, pre-formed peptide is released and then synthesis of new ACTH is stimulated to support the increased rate of release. In response to stress, peripheral and central signals are integrated by the pituitary to regulate ACTH and CGH release. Peripheral cytokine cascades, hypothalamic releasing factors, and intrapituitary cytokines act in a coordinate fashion to modulate the release of corticotrophic hormones. These cytokines include leukemia inhibitory factor (LIF) and interleukin (IL)-6, and may also act on the hypothalamus to regulate CRH release. See White and Ray, in Endocrinology (DeGroot and Jameson, eds) Vol. 1, pp. 221-233, W.B. Saunders, (2001).

### Corticotrophic Hormone Regulation of the Adrenal Cortex

The adrenal cortex produces three principal categories of steroid hormones. The mineralocorticoids, of which aldosterone is the most important, are produced in the zona glomerulosa, the outermost layer of the adrenal cortex, which comprises about 15% of the cortex. Directly beneath the zona glomerulosa is the zona fasciculata and then the zona reticularis, which together comprise approximately 75% of the cortex. The cells of the zona fasciculata are cholesterol-laden, and produce most of the glucocorticoid cortisol released into the circulatory system. The zona reticularis also produces cortisol, but primarily produces androgens such as dehydroepiandosterone (DHEA).

ACTH has a dramatic effect on adrenal function and architecture. Cortisol is released minutes after administration of ACTH, and within hours adrenal weight is increased. Prolonged corticotrophic stimulation causes both hypertrophy and hyperplasia of the cortex. Although cortisol is released minutes after stimulation, the primary regulation of steroid hormone secretion is at the level of steroid hormone synthesis. In the GC-producing cells of the cortex, receptor-mediated cyclic adenosine monophosphate (cAMP) production coupled to ACTH receptor activation results in activation of enzymes converting cholesterol to pregnenolone, the first step in cortisol synthesis. The rate-limiting enzymes for cortisol synthesis are regulated at the transcriptional level through increased cAMP, resulting in sustained activation of the synthetic machinery following hormone stimulation. Example 2 demonstrates the presence of CGH receptors in a cultured adrenal cell line through stimulation of cAMP production following treatment of the adrenal cell line with CGH. Thus CGH action in the adrenals produces activation of pathways important for adrenal cell function.

Conversely, when corticotrophic hormone concentrations are chronically low, a situation which can be engendered by extended or high-dose GC therapy, adrenal mass and steroidogenic activity decrease substantially. This condition, known as adrenal suppression, is a serious side-effect that may persist for weeks to months following GC therapy. During this prolonged recovery period, the adrenals are unable to mount an appropriate stress response under many circumstances. As described in example 4, treatment with CGH in conjunction with the glucocorticoid dexamethasone prevents the adrenal atrophy characteristic of extended glucocorticoid therapy. CGH used in conjunction with glucocorticoid therapy would be expected to reduce the prolonged suppression of the adrenals due to extended glucocorticoid therapy.

### Regulation of the Hypothalamic-Pituitary-Adrenal (HPA) Axis

The hypothalamus, pituitary, and adrenal gland form a neuroendocrine circuit whose principal function is the regulation of cortisol production. Cortisol exerts classical feedback regulation on this axis by decreasing the production of CRH and ACTH. CGH levels are likely to fall in parallel with ACTH due to their co-localization. Feedback-regulation of ACTH secretion by elevated cortisol has been described as having fast, intermediate, and slow components. Both fast and intermediate feedback appears to be mediated by inhibition of the release of existing CRH and ACTH, rather than by inhibition of their synthesis. Fast feedback blunts the ACTH response to weaker stimuli, but allows response to strong stimuli such as endotoxin and surgery. As glucocorticoid concentrations increase, particularly with extended glucocorticoid therapy, slow feedback produces decreased or absent synthesis of ACTH, and eventually unresponsiveness of the pituitary to the administration of CRH. See Miller and Chrousos, in Endocrinology and Metabolism (Felig and Frohman, eds), pp. 387-524, McGraw-Hill, (2001).

### Molecular Mechanisms of Glucocorticoid Action

Glucocorticoids can affect nearly all elements of inflammatory and immunologic responses. In general, GC's do not affect the condition or injury stimulating the primary response, but instead ameliorate the manifestations of the response to the initiating stressor. While it is generally accepted that basal GC levels are permissive of the stress response and may enhance it, elevated levels act to limit the response, thus contributing to the recovery. See Sapolsky et al., Endocr Rev 21: 55-89 (2000). Such interplay may serve to modulate the magnitude and duration of immune responses and prevent the overproduction of cytokines that can threaten homeostasis.

Glucocorticoids exert their effects on responsive cells by binding to a classical steroid hormone receptor, which, upon the binding of hormone, translocates to the nucleus of the cell and causes altered rates of transcription of target genes. The GC receptor (GR) is expressed throughout the body and is subject to very little feedback regulation. In inflammatory responses, GC's act to inhibit the production of acute-phase mediators of the immune response by repressing gene transcription. The most general effect of GC's is to inhibit the synthesis and release of cytokines and other inflammatory mediators that promote immune and inflammatory reactions. These include (but are not limited to) IL-1, IL-2, IL-3, IL-5, IL-6, IL-8, IL-12, TNFα tumor necrosis factor alpha), IFNγ (interferon gamma), RANTES (regulated on activation, expressed and secreted by normal T cells), nitric oxide, eicosanoids, collagenase, plasminogen activator, histamine, and elastase. See Sapolsky et al., Endocr Rev 21: 55-89 (2000).

GR-mediated gene repression results from inhibition of nuclear factor NF-κB, a well-characterized component of the pro-inflammatory signaling pathway. See Rothwarf and Karin, Sci STKE 1999: REI (1999). The NF-κB complex is made up of a family of transcription factors related to the Rel protein. Following stimulation, the NF-κB complex is activated in the cytoplasm by phosphorylation and subsequent degradation of the inhibitory IκB subunit. The functional p65-p50 dimer is translocated to the nucleus and binds specific sequences in the regulatory region of NF-κB target genes. It is thought that GR-mediated inhibition of cytokine signaling through NF-κB accounts for the anti-inflammatory and immunosuppressive effects of GC's. See Miesfeld, in Endocrinology (DeGroot and Jameson, eds) Vol. 2, pp. 1647-1654, W. B. Saunders, (2001).

The relative potency of GC's in eliciting therapeutic responses correlates with receptor-binding activities, and duration of action in the systemic circulation. The commonly used glucocorticoids are classified as short-acting, intermediate-acting, and long-acting. Cortisol, the natural human glucocorticoid produced in the adrenal cortex, is a short-acting glucocorticoid. Other examples include cortisone, prednisone, prednisolone, and methylprednisolone. Triamcinolone is an example of an intermediate-acting glucocorticoid. Betamethasone and Dexamethasone are examples of long-acting glucocorticoids. See Axelrod, in Endocrinology (DeGroot and Jameson, eds) Vol. 2, pp. 1671-1682, W.B. Saunders, (2001).

### The CGH Receptor

CGH exerts its effects through interaction with the thyroid-stimulating hormone (TSH), or thyrotropin, receptor. The TSH receptor (TSHR) is a member of the G-protein coupled, seven-transmembrane receptor superfamily. Activation of the TSH receptor leads to coupling with heterotrimeric G proteins, which evoke downstream cellular effects. The TSH receptor has been shown to interact with G proteins of subtypes Gₛ, G_{q}, G₁₂, and Gᵢ. In particular, interaction with Gₛ leads to activation of adenyl cyclase and increased levels of cAMP. See Laugwitz et al., Proc Natl Acad Sci USA 93: 116-20 (1996). Elevation of cAMP levels leads to activation of protein kinase A, a multi-potent protein kinase and transcription factor eliciting diverse cellular effects. See Bourne et al., Nature 349: 117-27 (1991).

The TSHR was originally identified in the thyroid as the principal activator of the thyroid gland, following exposure to the glycoprotein hormone, TSH. TSH release from the anterior pituitary stimulates the TSHR, resulting in secretion of thyroid hormone, stimulation of thyroid hormone synthesis, and cellular growth. TSH release is regulated by thyroid hormone levels, and is potently inhibited by elevated glucocorticoid levels. See, Utiger, in Endocrinology and Metabolism (Felig and Frohman, eds), pp. 261-347, McGraw-Hill, (2001).

Recently, the TSHR has been identified in many cell types not previously recognized, including cells of the immune system, brain, adipose, and reproductive organs. See, Example 3. These tissues are also targets of glucocorticoid action, suggesting a coordinate role for CGH and GC's as effectors of adrenal functions.

CGH is a potent activator of the TSHR. In adipose cells, sub-nanomolar levels of CGH stimulate release of free fatty acids (FFA). Compared to TSH, CGH stimulates the release of FFA at 10-fold lower molar concentrations.

### Actions of CGH in the Immune Response

Inflammation has been traditionally characterized by pain, swelling, redness, heat and loss of function. Inflammatory diseases can result from chronic or acute events, such as, but not limited to trauma, injury, and stress, and autoimmune conditions, and can be the result of, for example, surgery, infection, allergy, autoimmunity.

TSH receptors are found in many cells in the immune system, including targets of glucocorticoid action. These include monocyte/macrophages, T-cells, B-cells, dendritic cells, and polymorphonuclear leukocytes. See Example 3. Also see Bagriacik and Klein, J Immunol 164: 6158-65 (2000), and Kiss et al., Immunol Lett 55: 173-7 (1997). Flow cytometry using biotinylated CGH or TSH has been used to confirm expression of TSHR on the surface of these immune cell types (see Example 6. Also see Bagriacik and Klein, J Immunol 164: 6158-65, (2000). Activation of the TSHR has been shown to lead to increased cAMP in dendritic cells and T-cells, suggesting that these receptors are functional. Elevation of cAMP levels in a number of these cell types has been shown to inhibit the synthesis or secretion of several inflammatory cytokines, including IL-1, IL-6, IL-12, TNFα, and IFNγ. See Delgado and Ganea, J Biol Chem 274: 31930-40 (1999). In addition, the production of inflammatory mediators such as nitric oxide is suppressed by elevated cAMP in macrophages. See Delgado et al., Ann NY Acad Sci 897: 401-14 (1999). These actions parallel the biochemical events described above for glucocorticoid action in the immune system.

Production of TNFα by immune cells is a significant component of inflammatory events. Glucocorticoids act to suppress TNFα through inhibition of NF-κB, as described above. Activation of the TSHR and elevation of cAMP also results in inhibition of TNFα expression by inhibition of phosphorylation of transcription factor c-Jun, which is phosphorylated by JNK kinase. See Delgado et al., J Biol Chem 273: 31427-36 (1998). C-Jun phosphorylation is required for high-affinity interaction with DNA sequences in the TNFα promoter region. In the absence of stimulus, these DNA sequences are occupied by the cAMP responsive element binding protein (CREB) transcription factor, which may act as a negative regulator of transcription. CREB transcription factor is activated by phosphorylation by protein kinase A downstream of elevated cAMP, which has been shown to exert negative regulation on TNFα expression. See Delgado et al., J Biol Chem 273: 31427-36 (1998). Thus, CGH can act to suppress TNFα, an important inflammatory mediator, alone or in adjunctive therapy with GCs. As seen in Example 7, below, CGH treatment in vivo can indeed inhibit the production of TNFα in mice treated with a sublethal dose of endotoxin.

Elevated cAMP downstream of CGH binding to immune cells represses transcription of IRF-1, an important component of the ets-2 transcription factor complex. Ets-2 is required for high-level expression of IL-12, an important stimulator of T cell mediated inflammatory responses. See Ma et al., J Biol Chem 272: 10389-95 (1997). IL-12 participates in T cell activation and cytotoxic lymphocyte functions and promotes the differentiation of T helper (TH) cells into the TH1 subset. See Trinchieri, Int Rev Immunol 16: 365-96 (1998). Glucocorticoids inhibit IL-12 synthesis primarily through inhibition of transcription factor NF-κB. Thus CGH can be used to decrease the inflammatory response in a mammal by administering CGH alone, or in conjunction with glucocorticoids. The effects of this administration can be measured by a decrease in IL-12. Methods for measuring IL-12 levels are commonly known to one skilled in the art, and are commercially available.

In thymocytes and T cells, cAMP elevation produces stabilization of IκBα and subsequent impairment of NF-κB nuclear translocation. See Manna and Aggarwal, J Immunol 161: 2873-80 (1998). Other studies have reported the inhibition of NF-κB transcriptional activity by elevated cAMP via competitive mechanisms. Competition for limiting amounts of coactivator CREB binding protein (CREBP) between phosphorylated CREB and NF-κB is suggested to result in lower levels of NF-κB transcriptional activity. See Ollivier et al., J Biol Chem 271: 20828-35 (1996).

IL-10 is an anti-inflammatory cytokine, which down-regulates IL-12 and TNFα production. Moderate exposure of peripheral blood lymphocytes to GC's increases IL-10 production. See Franchimont et al., J Clin Endocrinol Metab 84: 2834-9 (1999). IL-10 release is inhibited only at the highest concentrations of GC's. IL-10 synthesis is increased by elevated cAMP. See Platzer et al., Eur J Immunol 29: 3098-104 (1999). This suggests a role for CGH alone and in combination with glucocorticoids in immune suppression.

The novel method of using CGH to treat inflammatory and immune diseases, as taught herein, can be used to target multiple components of the immune system. For example, as shown in Example 8, CGH treatment in vivo can suppress a delayed type hypersensitivity (DTH) reaction when administered either at the sensitization or at the challenge phase of the DTH response. The anti-inflammatory action of CGH is similar to that produced by the glucocorticoid dexamethasone in the DTH model of inflammation. Examples 7 and 8 demonstrate the potent anti-inflammatory action of CGH administered alone, and suggests that co-administration of CGH with glucocorticoids would provide a means of decreasing glucocorticoid dosages.

### Use of CGH to Potentiate or Replace Glucocorticoids

As a functional component of the HPA axis, CGH will find use as a therapeutic for the replacement of, or in conjunction with glucocorticoid therapy in all clinical indications in which glucocorticoids are beneficial

Glucocorticoids are among the most commonly used drugs. They are employed to treat many medical problems from minor skin conditions to life-threatening problems such as leukemia and organ transplant rejection. Clinical uses of CGH alone, or in conjunction with GC therapy include but are not limited to allergic disease, such as asthma, drug reactions and urticaria. Included also are arthritis, especially rheumatoid arthritis. Other uses include inflammatory gastrointestinal disease, such as, for example, inflammatory bowel disease and ulcerative colitis, subacute hepatic necrosis, and regional enteritis. Autoimmune diseases such as lupus erythematosus, Crohn's disease, and autoimmune hemolytic anemia are also included as indications for treatment with CGH alone or in combination with glucocorticoids. CGH may be especially beneficial for the treatment of transplant rejection, including but not limited to, kidney, liver, heart, and lung transplant. CGH would be especially beneficial for treatment of blood dyscrasias such as leukemia, multiple myeloma, idiopathic thrombocytopenic purpura, and acquired hemolytic anemia. Other diseases that would benefit from CGH include sarcoidosis, eye diseases treated with glucocorticoids, neurologic disease, renal disease, and malignant hyperthermia. In addition CGH can also be used to treat sepsis and multi-organ failure.

In addition, the polypeptides of the present invention can be used in diagnosis of inflammatory diseases. Such diagnoses can be performed by means of a kit that provides for forming a peptide-receptor complex, wherein CGH is the peptide, and TSHR is the receptor, and wherein inflammation is detected by measuring a decrease in a proinflammatory indicator.

### Formulations and Administration of CGH

CGH can be administered in conjunction with or in place of glucocorticoid treatment. This means that CGH is administered before, during or after administration of the steroid, as well as a stand-alone therapy. Treatment dosages should be titrated to optimize safety and efficacy. Methods for administration include intravenous, peritoneal intramuscular, and topical. Pharmaceutically acceptable carriers include but are not limited to water, saline, and buffers. Dosage ranges would ordinarily be expected from 0.1 μg to 0.1 mg per kilogram of body weight per day, with the exact dose determined by the clinician according to accepted standards, taking into account the nature and severity of the condition to be treated, patient traits, etc. Within this dosage range, a dose of 5 μg/kg/day can be used. Also within this range, a range from 5 μg/kg/day to 100 μg/kg/day can also be used. A useful dose to try initially would be 40 to 50 μg/kg per day. However, the doses may be higher or lower as can be determined by a medical doctor with ordinary skill in the art. For a complete discussion of drug formulations and dosage ranges see Remington's Pharmaceutical Sciences, 17^{th} Ed., (Mack Publishing Co., Easton, Penn., 1990), and Goodman and Gilman's: The Pharmacological Bases of Therapeutics, 9^{th} Ed. (Pergamon Press 1996).

For pharmaceutical use, the proteins of the present invention can be administered orally, rectally, parenterally (particularly intravenous or subcutaneous), intracisternally, intravaginally, intraperitoneally, topically (as powders, ointments, drops or transdermal patch) bucally, or as a pulmonary or nasal inhalant. Intravenous administration will be by bolus injection or infusion over a typical period of one to several hours. In general, pharmaceutical formulations will include a CGH protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Doses of CGH polypeptide will generally be administered on a daily to weekly schedule. Determination of dose is within the level of ordinary skill in the art. The proteins may be administered for acute or chronic treatment, over several days to several months or years. In general, a therapeutically effective amount of CGH is an amount sufficient to produce a clinically significant change in an inflammatory condition.

### Benefits of CGH Administration

While GC's are effective ubiquitous physiological regulators, GC therapy can have severe adverse side effects. Therefore, it is highly desirable to lower the effective dosage of GC's in any therapeutic protocol. Adjunctive therapies that potentiate, or enhance, or even replace glucocorticoid action will allow the use of lower doses of GC's. The discovery of CGH as a mediator of immune suppression leads to the ability to administer CGH with or without GC's, or and thus produce equivalent therapeutic efficacy with lower GC dosage.

Example 9 demonstrates that 4 weeks of daily CGH treatment of normal mice did not lead to any measurable alterations in the lymphoid cell populations. Similar treatment with GC's leads to dramatic decreases in several lymphoid cell populations, particularly in the thymus, the site of T-lymphocyte maturation. As a result, treatment with GC's increases risk of infections, including bacterial, viral, fungal, and parasitic. See Chrousos, in Endocrinology and Metabolism (Felig and Frohman, eds), pp. 609-632, McGraw-Hill (2001). Co-administration of CGH to reduce GC dosage or substitute therapy with CGH alone is expected to reduce the risk of these infections.

Some adverse consequences of GC therapy are related more to the dose than to the duration of treatment. Avascular or ischemic necrosis of bone is a common side-effect of GC therapy and is thought to be consequence of dosage. See Chrousos, in Endocrinology and Metabolism (Felig and Frohman, eds), pp. 609-632, McGraw-Hill (2001). There is evidence that steroid-induced cataracts, a significant risk in GC treated patients, are due to high local concentrations of glucocorticoid in these individuals. The discovery of CGH as a modulator of inflammation permits GC dosage to be reduced, with a concomitant reduction in side effects of GC administration.

Other adverse consequences of GC therapy may be ameliorated by CGH co-administration. Osteoporosis is a major limiting factor in long-term glucocorticoid therapy. Glucocorticoids are thought to act both on bone-forming cells, in part by producing apoptosis, and on osteoclasts, by stimulating bone resorption. High dosages of GC's are known to inhibit intestinal calcium absorption. See Singer, in Endocrinology and Metabolism (Felig and Frohman, eds), pp. 1179-1219, McGraw-Hill (2001). Reduction of GC dosage has been investigated as a means of minimizing the adverse consequences in bone of long-term GC use, and the use of the lowest possible therapeutic dose is strongly encouraged. See Chrousos, in Endocrinology and Metabolism (Felig and Frohman, eds), pp. 609-632, McGraw-Hill (2001). The presence of CGH receptors in osteoblasts (Example 3), the bone-forming cells, also suggests CGH is protective to GC-mediated inhibition of bone formation. Thus, in bone, activation of the CGH receptor would increase cAMP, which stimulates differentiated functions in bone cells, thus inhibiting the pro-apoptotic effect of glucocorticoids. See Siddhanti and Quarles, J Cell Biochem 55: 310-20 (1994).

Additional side effects of glucocorticoid administration that would diminish with CGH co-administration include obesity, corticosteroid-induced psychosis, gastrointestinal hemorrhage, and benign intracranial hypertension.

Additionally, as indicated above, and taught herein, the anti-inflammatory effects of CGH will allow CGH administration to replace glucocorticoid therapy entirely.

CGH can also be used to treat arthritis as either stand-alone therapy, or in conjunction with a glucocorticoid, such as, for example, dexamethasone or prednisone. The effects of CGH can be measured in a in vivo model for collagen induced arthritis. See Tanaka, Y. et al., Inflamm. Res. 45:283-88, 1996.

### Use of CGH to Reduce HPA Suppression

Feedback regulation of the HPA axis is described above. During GC therapy, adrenal cortex function can become significantly suppressed. Prolonged suppression can cause extreme atrophy of the adrenal cortex, due to privation of ACTH and its growth-stimulating activity. Recovery from such feedback suppression occurs slowly and is always an important consideration following the withdrawal of glucocorticoid therapy. Functional recovery of the adrenal cortex to the extent that a patient is able to mount an appropriate stress response following chronic GC treatment takes from 1-12 months. As a result, glucocorticoid administration is typically continued at maintenance levels for several months following the end of therapeutic treatment. See Chrousos, in Endocrinology and Metabolism (Felig and Frohman, eds), pp. 609-632, McGraw-Hill (2001).

The time course of recovery of the adrenal cortex correlates with the total duration of previous GC therapy as well as the total glucocorticoid dose. The rate of recovery is also determined by the doses given when the GC is being tapered as well as the doses administered during the initial phases of the treatment. Recovery of the adrenal axis generally requires ACTH levels beyond the normal physiological range to reverse the atrophy associated with adrenal suppression. Elevated ACTH levels are typically seen for a period of months following the cessation of GC therapy, before sufficient basal cortisol levels lower ACTH levels through feedback inhibition. However, even after ACTH levels return to the normal range, exposure of the adrenals to elevated ACTH following a significant stressor results in a blunted response. For this reason, low dose GC administration is continued to ensure that patients can mount an adequate stress response. The use of ACTH to stimulate endogenous GC production as a substitute for exogenous GC administration has been extensively investigated. See Axelrod, in Textbook of Rheumatology (Kelley et al., eds), WB Saunders (1993). The use of ACTH dramatically reduces adrenal cortex suppression, and in some cases, results in an overactive and hyperplastic state. However, due to the concomitant mineralocorticoid and androgen stimulating properties of ACTH administration, ACTH is not a preferred treatment modality. The use of ACTH to reverse adrenal suppression following cessation of exogenous GC treatment has also been studied. The administration of ACTH does not reverse the development or course of adrenal insufficiency. See Chrousos, in Endocrinology and Metabolism (Felig and Frohman, eds), pp. 609-632, McGraw-Hill (2001). To date, there is no method for hastening a return to normal HPA function once inhibition has resulted from glucocorticoid therapy.

Adrenal suppression is assessed clinically with standard practices. See Axelrod, in Textbook of Rheumatology (Kelley et al., eds) WB Saunders (1993). Glucocorticoids are withdrawn for approximately 24 hours, and a measured dose of ACTH is given. The relative increase in cortisol from baseline is measured at specific times following ACTH administration to assess the ability of the adrenals to respond adequately to a significant stress-related event. Due to fluctuations in basal cortisol levels, adrenal sufficiency is determined by increases in cortisol production, not by the absolute measured level.

The discovery of the corticotrophic properties of CGH offers a novel method of preventing adrenal suppression. Co-administration of CGH in glucocorticoid therapy can reduce or prevent adrenal atrophy, and in turn, adrenal suppression. CGH acts upon the adrenal cortex through activation of TSH receptors in the adrenal cortex. This stimulates the production of cAMP in the cortex, which is necessary for the maintenance of normal cortical function.

Example 4 demonstrates the potency of cortical stimulation produced by CGH. Profound adrenal hypertrophy was observed in mice following introduction of adenoviral vectors expressing CGH, leading to overexpression and secretion of CGH from the liver of these animals. The hypertrophy was apparent in the inner cortical layers, the zona fasciculata and the zona reticularis. These two zones of the cortex, as described above, are responsible for the synthesis and release of cortisol from the adrenals. Example 4 also describes the potency of cortical stimulation produced by chronic injection of CGH protein. A significant increase in adrenal weight was demonstrated after two weeks of daily injections of recombinant CGH to normal female mice. These two experiments suggest that CGH stimulation of the adrenal cortex is a component of HPA axis regulation. Further, in the absence of ACTH, introduced CGH will provide stimulatory signals needed by the adrenal cortex to maintain cortical function. As taught in Example 4, CGH used in conjunction with glucocorticoids prevents adrenal atrophy as demonstrated by the prevention of loss of adrenal weight seen following treatment with glucocorticoid alone. Use of CGH with glucocorticoids can reduce adrenal suppression by two mechanisms. First, co-administration of CGH can allow the use of lower total doses of glucocorticoids, which can in turn result in less severe suppression, as described above. Second, the stimulatory effect of CGH on the adrenal cortex can ameliorate the atrophy of the cortex, preventing the long-term suppression of the adrenal gland, and restoring HPA axis response to significant stressors.

The invention is further illustrated by the following non-limiting examples.

### EXAMPLE 1

### CGH is Expressed in Corticotrophs

Summary: The cell-specific localization of CGH expression in the anterior pituitary was evaluated in two stages. First, double in situ hybridization was used to demonstrate the co-expression of GPHA2 and GPHB5 mRNAs in the same subset of cells in the anterior pituitary. Next, the identity of these cells was evaluated using double immunohistochemical methods to stain for the localization of GPHB5 protein relative to protein markers for different cell populations in the anterior pituitary. These markers included growth hormone (a marker for somatotrophs), follicle-stimulating hormone (gonadotrophs), luteinizing hormone (gonadotrophs), thyroid-stimulating hormone (thyrotrophs), adrenocorticotrophic hormone (corticotrophs), prolactin (mammotrophs) and S-100 protein (follicular stellate and dendritic cells). GPHB5 protein was found to co-localize with adrenocorticotrophic hormone, showing that it was produced by corticotrophs. GPHB5 protein was not found to co-localize with any of the other markers. Taken together, the immunohistochemical data and the in situ data described above show that the heterodimeric glycoprotein hormone CGH is produced by corticotrophs.

A. Identification of Cells Expressing GPHA2 and GPHB5 Using In Situ Hybridization.

Human pituitaries were screened for GPHA2 and GPHB5 expression by in situ hybridization. The tissues were fixed in 10% buffered formalin and embedded in paraffin blocks using standard techniques. Tissues were sectioned at 4 to 8 microns, and the sections were prepared using a standard protocol. Briefly, tissue sections were deparaffinized with HistoClear (National Diagnostics, Atlanta, Ga.) and then dehydrated with ethanol. Next they were digested with Proteinase K (50 μg/ml) (Boehringer Diagnostics, Indianapolis, Ind.) at 37° C. for 3 to 10 minutes. This step was followed by acetylation and re-hydration of the tissues.

Using oligonucleotides specific for GPHB5 sequences, a polymerase-chain-reaction-based in situ method was used to visualize GPHB5 mRNA with a FITC detection system, which gives a green signal. Following this reaction, the same slide was subjected to a standard in situ hybridization protocol using a probe designed against the human GPHA2 sequence. T7 RNA polymerase was used with a linearized plasmid template containing the entire coding domain and the 3′UTR of GPHA2 to generate an antisense probe. The probe was labeled with digoxigenin (Boehringer, Ingelheim, Germany) using an In Vitro Transcription System kit (Promega, Madison, Wis.) following the manufacturer's instructions. The digoxigenin-labeled GPHA2 probe was added to the slides at a concentration of 1 to 5 pmol/ml for 12 to 16 hours at 60° C. Slides were subsequently washed in 2×SSC and 0.1×SSC at 55° C. The signals were amplified using tyramide signal amplification (TSA, in situ indirect kit; NEN, PerkinElmer Life Sciences, Boston, Mass.) and visualized with Texas Red following the manufacturer's instructions. The slides were then counter-stained with hematoxylin (Vector Laboratories, Burlingame, Calif.) and evaluated microscopically.

Results: A subset of scattered cells in the anterior pituitary show both green and red signal, indicating that they were positive for both GPHB5 and GPHA2 mRNA expression. There are few or no cells that express only one of the two messages.

B. Immunohistochemical Double Staining of GPHB5 vs. Markers for Anterior Pituitary Cell Types

Human anterior pituitaries were screened using antibodies against GPHB5 and a variety of cell-type-specific markers to determine which cell types express GPHB5 protein. Double immunostains were performed for GPHB5 vs. growth hormone (GH; a marker for somatotrophs), follicle-stimulating hormone (FSH; gonadotrophs), luteinizing hormone (LH; gonadotrophs), thyroid-stimulating hormone (TSH; thyrotrophs), adrenocorticotrophic hormone (ACTH; corticotrophs), prolactin (PRL; mammotrophs) and S-100 protein (follicular stellate and dendritic cells).

Sandwich technique immunohistochemistry was applied in this study, using two primary antibodies (anti-GPHB5 and antibodies against one of the marker proteins) and two detection systems: immunoperoxidase (IP) with Diaminobenzidine (DAB) (Ventana Bio Tek, Tucson, Ariz.), leading to a brown signal indicating the presence of GPHB5, and alkaline phosphatase (AP) with BioTek Red, (Ventana Bio Tek) leading to a red signal indicating the presence of the marker protein in question.

The experiments were performed on sections of a human pituitary gland taken from a 24-year-old male who died of a gunshot wound (tissue block internal reference number H01.2075). The tissue was fixed in 10% buffered formalin and embedded in paraffin blocks using standard techniques. The tissue was sectioned at 4 to 8 microns, and the sections were prepared using a standard protocol.

Reagents and Protocol:

Normal goat blocking serum (ChemMate, CMS/Fisher: Cat #: 028-337).

Primary antibodies: a) Rabbit anti-human GPHB5 protein (produced in-house, internal reference number E3039), working dilution: 1:3200. b) Rabbit anti-human GH (Zymed Laboratories, South San Francisco, Calif.) Cat. No. 18-0090), working dilution: 1:25. c) Mouse anti-human FSH (Zymed, Cat. No. 18-0020), working dilution: 1:50. d) Mouse anti-human LH (Zymed, Cat. No. 18-0037), working dilution: 1:50. e) Mouse anti-human TSH (Zymed, Cat. No. 18-0051), working dilution: 1:50. f) Rabbit anti-human ACTH (Zymed, Cat. No. 18-0087), working dilution: 1:50. g) Rabbit anti-PRL (Zymed, Cat. No. 18-0086), working dilution: 1:50. h) Rabbit anti-S-100 protein (Zymed, Cat. No. 18-0046), working dilution: 1:1000 and 1:2000.

Secondary antibodies: a) Biotinylated goat anti rabbit IgG (Vector, Cat. No: BA-1000), working solution: 7.5 μg/l, diluted in PBS with 2% normal goat serum. b) Biotinylated goat anti mouse IgG (Vector, Cat. No: BA-9200), working solution: 7.5 μg/l, diluted in PBS with 2% normal goat serum and 2% non-fat dried milk.

Detection reagents: a) DAB Detection Kit (Ventana Bio Tek Systems, Tucson, Ariz. Catalog No: SDK2502). b) AP Detection Kit (Ventana Catalog No: SDK306).

Method: TechMate 500 autoimmunstainer (Biotech/Ventana), IP-AP protocol with modifications. Avidin/Biotin block following heat-induced epitope retrieval.

Summary of Results:

Positive staining was seen for GPHB5 and all other primary antibodies. GPHB5 was found to co-localize only with ACTH, and not with FSH, GH, LH, PL and TSH. GPHB5/S-100 staining was less than optimal, but GPHB5 and S-100 co-localization was not indicated. GPHB5 staining was seen in the majority of ACTH-producing pituicytes. There are few if any cells producing GPHB5 that do not also express ACTH.

### EXAMPLE 2

### CGH Activation of Adrenal Cortex Cells Results in cAMP Production

Summary: A human adrenal cortex cell line, NCI-H295R, was used to study signal transduction of CGH. NCI-H295R was transduced with recombinant adenovirus containing a reporter construct, a firefly luciferase gene under the control of cAMP response element (CRE) enhancer sequences. This assay system detects cAMP-mediated gene induction downstream of activation of G-coupled GPCR's (G-protein coupled receptors). Treatment of NCI-H295 with purified CGH heterodimer protein produced a dose-dependent induction of luciferase activity equal to or higher than that induced by 110 M forskolin, a constitutive inducer of adenyl cyclase. Typically, CGH elicited a maximal response of 15-40-fold luciferase induction above control media. These results demonstrate CGH signaling through a GPCR in the adrenal cortex and the production of cAMP.

Experimental Procedure.

NCI-H295R cells were obtained from the ATCC (CRL-2128, Manassas, Va.) and cultured in growth medium as follows: 1:1 mixture of Dulbecco's modified Eagle's medium and Ham's F12 medium with L-glutamine (D-MEM/F-12; GIBCO, cat.#11320-033) containing 25 mM HEPES buffer (GIBCO, Invitrogen, Carlsbad, Calif., cat.#15630-080), 1 mM sodium pyruvate (GIBCO, cat.#11360-070), 1% ITS+1 media supplement (Sigma St. Louis, Mo. cat#12521) and 2.5% Nu-Serum I (BD Biosciences, Lexington, Ky. cat.#355100). Cells were cultured at 37° C. in a 5% CO₂ humidified incubator. One or two days before assaying, cells were seeded at 20,000 cells per well in a 96-well white opaque/clear bottom plate (BD Biosciences, cat.#356650). One day before assay, cells were transduced with AV KZ55, an adenovirus vector containing KZ55, a CRE-driven luciferase reporter cassette, at 5000 particles per cell. Following overnight incubation, the cells were rinsed once with assay medium (D-MEM/F-12 supplemented with 0.1% bovine serum albumin, ICN Biomedicals, Inc., Aurora, Ohio, cat.#103700), followed by incubation for four hours at 37° C. in assay medium to which test protein had been added. The plate was then washed with phosphate buffered saline (GIBCO, cat. #20012-027).

Promega's Luciferase Assay System (Promega, Madison, Wis., cat. #E1500) was used to process the treated cells. Cell lysis buffer, 25 μl/well, was added to each well and incubated at room temperature for 15 minutes. Luciferase activity was measured on a microplate luminometer (PerkinElmer Life Sciences, Inc., model LB 96V2R) following automated injection of luciferase assay substrate.

### EXAMPLE 3

### Distribution of TSH Receptor Gene Expression

We surveyed RNA samples for TSHR transcript using reverse transcriptase polymerase chain reaction (RT-PCR) amplification. Using standard procedures, RNA samples were isolated from tissues and cell lines, and RT-PCR was run with two separate pairs of primers. The first primer pair includes the forward primer (5′TCAGAAGAAAATCAGAGGAATC) (SEQ ID NO:8) and the reverse primer (5′GGGACGTTCAGTAGCGGTTGTAG) (SEQ ID NO:9), which amplify a 487 bp product. The amplified product spans an intron to control for signal arising from genomic DNA contamination. The second primer pair includes the forward primer (5′CTGCCCATGGACACCGAGAC) (SEQ ID NO:10) and the reverse primer (5′CCGTTTGCATATACTCTTCTGAG) (SEQ ID NO:11) and amplifies a 576 bp product. Additionally, TSHR expression was assessed from data in the published literature. Results are described below.

A. TSH Receptor in Immune Related Cells.

TSH-R is expressed in human CD14+ monocytes (decreasing expression after activation), in the human monocytic cell lines THP-1 and PMA-activated HL60 (but not in U937), in resting (but not activated) human NK cells, in human “resting” CD3+ (primarily CD4+) T cells, and in human B cells and B cell lines. Among mouse immune cell subsets, we have found that mTSH-R is expressed in CD4+ but not CD8+ T cells (decreasing with activation), in B cells (decreasing slightly with activation), and in an IFNγ-activated mouse macrophage line, J774.

Additionally, TSHR transcript has also been shown to be present in lymphocytes (Szkudlinski M. W., Fremont V., Ronin C., Weintraub B. D., (2002) Physiol Rev 82: 473-502) and other immune related cell types (Bagriacik E U, and Klein J R, (2000) J Immunol 164: 6158-65).

B. TSH Receptor in Adrenal Gland.

RNA from the adrenal cortex carcinoma cell line H295R along with RNA isolated from several adult human normal adrenal glands were found positive for TSHR. Published literature also documents TSHR transcript in the adrenal gland (Dutton C. M., Joba W., Spitzweg C., Heufelder A. E., Bahn R. S., (1997) Thyroid 6: 879-84).

C. TSH Receptor in a Wide Variety of Cells and Tissue Types.

Extensive panels of RNAs were screened for TSHR and positive expression was found in thyroid, adrenal gland, kidney, brain, skeletal muscle, testis, liver, osteoblast, aortic smooth muscle, ovary, adipocytes, retina, salivary gland, and digestive tract. Similarly, the published literature documents TSHR expression in thyroid, kidney, thymus, adrenal gland, brain, retroocular fibroblasts, neuronal cells and astrocytes (Szkudlinski M. W., Fremont V., Ronin C., Weintraub B. D., (2002) Physiol Rev 82: 473-502 and Dutton C. M., Joba W., Spitzweg C., Heufelder A. E., Bahn R. S., (1997) Thyroid 6: 879-84).

### EXAMPLE 4

### In Vivo Stimulation of Adrenal Cortex by CGH

Summary: Mice were exposed to CGH through infection with adenovirus particles expressing GPHA2 and GPHB5, leading to overexpression and secretion of CGH from the liver of these animals. Profound adrenal hypertrophy and vacuolization were observed in mice sacrificed three weeks after adenoviral infection. The hypertrophy was apparent in the inner cortical layers, the zona fasciculata and the zona reticularis. Similarly, mice were exposed to CGH through intraperitoneal injection of recombinant CGH protein alone, recombinant CGH protein along with the glucocorticoid Dexamethasone (Dex), Dex alone, or PBS alone daily for two weeks. Significant gain in adrenal weight was demonstrated in female mice after chronic treatment with CGH or CGH along with Dex.

A. Generation of GPHB5 and GPHA2 Expressing Recombinant Adenovirus.

The protein coding regions of GPHA2 and GPHB5 were amplified by PCR using primers that added FseI and AscI restriction sites at the 5′ and 3′ termini respectively. PCR primers were used with the templates containing the full-length GPHA2 and GPHB5 cDNAs in standard PCR reactions. The PCR reaction products were loaded onto a 1.2% (low melt) SeaPlaque GTG (FMC, Rockland, Me.) gel in TAE buffer. The products were excised from the gel and purified using the QIAquick®PCR Purification Kit gel cleanup kit as per kit instructions (Qiagen, Valencia, Calif.). The PCR products were then digested with FseI-AscI, phenol/chloroform extracted, EtOH precipitated, and rehydrated in 20 uL TE (Tris/EDTA pH 8). The products were then ligated into the FseI-AscI sites of the vector pMT12-8 and transformed into DH10B cells by electroporation. Clones containing the appropriate inserts were identified by plasmid DNA miniprep followed by digestion with FseI-AscI, and the constructions verified by DNA sequencing. DNA was prepared using a commercially available kit (Qiagen, Inc.) The GPHA2 and GPHB5 cDNAs were released from the pMT12-8 vector using FseI and AscI enzymes. The cDNAs were isolated on a 1.2% low melt gel, the gel slices melted at 70° C., extracted twice with an equal volume of Tris-buffered phenol, and EtOH precipitated. The DNAs were resuspended in 10 uL of water.

The GPHA2 and the GPHB5 recombinant adenoviruses were prepared using different vectors. The GPHA2 cDNA was ligated into pACCMV shuttle vector (Microbix Biosystems, Inc. Ontario, Canada) in which the polylinker had been modified to include FseI and AscI sites and transformed into E. coli host cells (Electromax DH10B™ cells; obtained from Life Technologies, Inc., Gaithersburg, Md.) by electroporation. Clones containing GPHA2 inserts were identified by plasmid DNA miniprep followed by digestion with FseI and AscI. A large-scale preparation of DNA was made for transfection. The GPHA2-containing shuttle vectors were co-transfected with EI-deleted, adenovirus vector pJM17 (Microbix Biosystems, Inc.) into 293A cells (Quantum Biotechnologies, Inc. Montreal, QC. Canada) that express the adenovirus E I gene. The DNA was diluted up to a total volume of 50 ul with sterile HBS (150 mM NaCl, 20 mM HEPES). In a separate tube, 20 uL DOTAP (Boehringer-Ingelheim, 1 mg/ml) was diluted to a total volume of 100 ul with HBS. The DNA was added to the DOTAP, mixed gently by pipetting up and down, and left at room temperature for 15 minutes. The media was removed from the 293A cells and washed with 5 ml serum-free MEMalpha containing 1 mM sodium pyruvate, 0.1 mM MEM non-essential amino acids and 25 mM HEPES buffer (all from Life Technologies, Inc.). 5 ml of serum-free MEM was added to the 293A cells and held at 37° C. The DNA/lipid mixture was added drop-wise to the T25 flask of 293A cells, mixed gently, and incubated at 37° C. for 4 hours. After 4 hours the media containing the DNA/lipid mixture was aspirated off and replaced with 5 ml complete MEM containing 5% fetal bovine serum.

The 293A cells were maintained for 2-4 weeks before recombination of the endogenous viral sequences and the transfected viral vector resulted in the production of infectious viral particles. Within 5 days of recombination, propagation of infectious virus produced lysis of the culture monolayer. The medium containing the viral lysate was collected and any remaining intact cells were lysed by repeated freeze/thaw cycles and the cell debris was pelleted by centrifugation.

The viral lysate was then plaque-purified according to the method of Becker et al., Meth. Cell Biol. 43:161-189, 1994. Briefly, serial dilutions were prepared in DMEM containing 10% fetal bovine serum and 100 U/ml penicillin/streptomycin, added to monolayers of 293 cells, and incubated at 37° C. for one hour. A melted 1.3% agarose/water solution was mixed with 2×DMEM (containing 4% FBS, 200 U/ml penicillin/streptomycin, 0.5 ug/ml fungizone and 30 mg/ml phenol red), and 6 ml of the mixture was added to the virus-infected 293 cells. Plaques were visible within 7-10 days. Single plaques were isolated, and the presence of the GPHA2 insert was verified by PCR. One plaque that had the expected size PCR product was used to do a primary amplification.

The GPHB5 adenoviral construction was produced in a second vector system, pAdTrack CMV (He, T-C. et al., PNAS 95:2509-2514, 1998). This vector contains the Green Fluorescent Protein (GFP) marker gene, and was first modified by replacing the promoter and polyadenylation sequences of the GFP gene with SV40 and human growth hormone sequences, respectively. In addition, the native polylinker was replaced with FseI, EcoRV, and AscI sites. This modified form of pAdTrack CMV was named pZyTrack. Ligation was performed using the Fast-Link® DNA ligation and screening kit (Epicentre Technologies, Madison, Wis.). Clones containing GPHB5 were identified by digestion of mini prep DNA with FseI-AscI. In order to linearize the plasmid, approximately 5 μg of the pZyTrack GPHB5 plasmid was digested with PmeI. Approximately 1 ug of the linearized plasmid was cotransformed with 200 ng of supercoiled pAdEasy (He et al., supra.) into BJ5183 cells. The co-transformation was done using a Bio-Rad Gene Pulser at 2.5 kV, 200 ohms and 25 mFa. The entire co-transformation was plated on 4 LB plates containing 25 ug/ml kanamycin. The smallest colonies were picked and expanded in LB/kanamycin and recombinant adenovirus DNA identified by standard DNA miniprep procedures. Digestion of the recombinant adenovirus DNA with FseI-AscI confirmed the presence of GPHB5. The recombinant adenovirus miniprep DNA was transformed into DH10B competent cells and DNA prepared using a Qiagen maxi prep kit as per kit instructions.

Approximately 5 ug of recombinant adenoviral DNA was digested with PacI enzyme (New England Biolabs, Beverly, Mass.) for 3 hours at 37° C. in a reaction volume of 100 uL containing 20-30 U of PacI. The digested DNA was extracted twice with an equal volume of phenol/chloroform and precipitated with ethanol. The DNA pellet was resuspended in 10 uL distilled water. A T25 flask of QBI-293A cells (Quantum Biotechnologies, Inc. Montreal, Qc. Canada), inoculated the day before and grown to 60-70% confluence, were transfected with the PacI digested DNA. The PacI-digested DNA was diluted up to a total volume of 50 uL with sterile HBS (150 mM NaCl, 20 mM HEPES). In a separate tube, 20 uL DOTAP (Boehringer-Ingelheim, 1 mg/ml) was diluted to a total volume of 100 uL with HBS. The DNA was added to the DOTAP, mixed gently by pipetting up and down, and left at room temperature for 15 minutes. The media was removed from the 293A cells and washed with 5 ml serum-free MEMalpha (Gibco-Invitrogen) containing 1 mM Sodium Pyruvate (Gibco-Invitrogen), 0.1 mM MEM non-essential amino acids (Gibco-Invitrogen) and 25 mM HEPES buffer (Gibco-Invitrogen). 5 mL of serum-free MEM was added to the 293A cells and held at 37° C. The DNA/lipid mixture was added drop-wise to the T25 flask of 293A cells, mixed gently and incubated at 37° C. for 4 hours. After 4 h the media containing the DNA/lipid mixture was aspirated off and replaced with 5 ml complete MEM containing 5% fetal bovine serum. The transfected cells were monitored for GFP expression and plaque formation. Seven days after transfection of 293A cells with the recombinant adenoviral DNA, the cells expressed the GFP protein and began to form visible plaques. The crude viral lysate was collected by using a cell scraper to collect the 293A cells. The lysate was transferred to a 50 mL conical tube. To release most of the virus particles from the cells, three freeze/thaw cycles were done in a dry ice/ethanol bath and a 37° waterbath. The crude lysate was amplified to obtain a working stock of GPHB5 recombinant adenoviral lysate.

B. Amplification and Purification of GPHA2 and GPHB5 Recombinant Adenoviruses.

200 uL of crude recombinant adenoviral lysate was added to each of ten nearly confluent 10 cm plates. The infections were monitored for 48 to 72 hours for cytopathic effect (CPE) under the white light microscope or expression of GFP (GPHB5 virus) under the fluorescent microscope. When all of the 293A cells exhibited CPE, a stock lysate was collected and freeze/thaw cycles performed.

Secondary amplification of the recombinant adenoviruses was achieved with twenty 15-cm tissue culture dishes of 293A cells at 80-90% confluency. Media volume was reduced to 20 mls of 5% MEM and each dish was inoculated with 300-500 uL of amplified stock viral lysate. Complete lysis of the cultures was observed after 48 hours and the lysate collected into 250 ml polypropylene centrifuge bottles. NP-40 detergent was added to a final concentration of 0.5% to ensure complete cell lysis. Bottles were placed on a rotating platform for 10 minutes with rapid agitation. The debris was pelleted by centrifugation at 20,000×G for 15 minutes. The supernatant was transferred to 250-ml polycarbonate centrifuge bottles, and 0.5 volumes of 20% PEG8000/2.5M NaCl solution were added. The bottles were shaken overnight on ice. The bottles were centrifuged at 20,000×G for 15 minutes, and the supernatant discarded. The viral precipitate from 2 bottles was resuspended in 2.5 ml PBS. The resulting virus solution was placed in 2-ml microcentrifuge tubes and centrifuged at 14,000×G for 10 minutes to remove any additional cell debris. The supernatant from the 2-ml microcentrifuge tubes was transferred into a 15-ml polypropylene snap-cap tube and adjusted to a density of 1.34 g/ml with cesium chloride (CsCl). The solution was transferred to 3.2 ml polycarbonate thick-walled centrifuge tubes (Beckman) and spun at (348,000×G) for 3-4 hours at 25° C. The virus formed a white band. Using wide-bore pipette tips, the virus band was collected.

Pharmacia PD-10 columns prepacked with Sephadex G-25M (Pfizer-Pharmacia, New York, N.Y.) were used to desalt the virus preparation. The column was equilibrated with 20 mL of PBS. The virus was loaded and allowed to run into the column. 5 mL of PBS was added to the column and fractions of 8-10 drops collected. The optical densities of 1:50 dilutions of each fraction were determined at 260 nm on a spectrophotometer. A clear absorbance peak was present between fractions 7-12. These fractions were pooled and the optical density (OD) of a 1:10 dilution determined. A formula is used to convert OD into virus concentration: (OD at 260 nm)(10)(1.1×10¹²)=virions/mL. The GPHB5 recombinant adenovirus concentration was 1.99×10¹² virions/mL. The GPHA2 recombinant adenovirus concentration was 6.1×10¹² virions/ml. Glycerol was added to the purified virus to a final concentration of 15%, and stored in aliquots at −80° C.

C. Adenoviral Infection of Mice and Results of Treatment.

Each group consisted of eight female C57BL6 mice. 7.5×10¹¹ particles each of GPHA2- and GPHB5-expressing adenovirus were administered by tail vein injection to the experimental group, while 1.5×10¹¹ particles of adenovirus expressing a parental vector alone were administered to the control group. Animals were sacrificed on day 20 following the injection and tissues were evaluated by a pathologist. Treatment-related effects were observed in the adrenal glands of all eight mice in the experimental group; no effects were observed in the adrenal glands of the control group. The CGH-induced histomorphological changes of the inner adrenal cortical cells included profound hypertrophy and uniformly finely, foamy vacuolization.

Intraperitoneal Injection of Recombinant CGH and Results of Treatment.

Sixteen C57BL/6 female mice at 8 weeks of age were separated into four groups. The first group received daily injections of 0.25 mg/kg of recombinant CGH protein intraperitoneally. The second group received daily injections of PBS using the same procedure. The third group received daily injections of 0.25 mg/kg of CGH plus 0.05 mg/kg Dex and the final group received 0.5 mg/kg Dex, alone. Animals were sacrificed on day 15 and the adrenal glands were isolated and weighed. Results are shown in Table 1. Example 5 describes the expression and purification of recombinant CGH protein used in this experiment.

TABLE 1
Significant increase in adrenal weight after chronic CGH treatment.

| | | | Average | |
|---|---|---|---|---|
| | | | adrenal | |
| Group | | Number of | weight/100 g | |
| Number | Treatment | Mice | body weight | P value |
| 1 | 0.25 mg/kg | 4 | 22.26 +/− 0.99 | Group 1 and 2 |
| | CGH | | | |
| 2 | PBS | 4 | 15.32 +/− 2.21 | 0.0012 |
| 3 | 0.25 mg/kg | 4 | 17.66 +/− 1.86 | Group 3 and 4 |
| | CGH + 0.5 mg/kg | | | |
| | Dex | | | |
| 4 | 0.5 mg/kg Dex | 4 | 13.12 +/− 0.88 | 0.0046 |

### EXAMPLE 5

### Expression and Purification of Recombinant CGH

A. Generation of CHO 180.

The CGH-producing cell line CHO 180 was generated in two stages. A construct expressing GPHA2, GPHB5 and drug resistance (dihydrofolate reductase) from the CMV promoter was transfected to protein-free CHO DG44 cells (PF CHO) by electroporation. The resulting pool was selected and amplified using methotrexate. Early analysis indicated a high level of GPHA2 expression, but a low level of GPHB5 expression. Therefore, a second construct expressing GPHB5 from the CMV promoter and zeocin resistance from the SV-40 promoter was transfected into the selected, amplified pool by electroporation. After zeocin selection, the final pool (CHO 180) expressed significant levels of both GPHA2 and GPHB5; the proteins were secreted as the non-covalent heterodimer, CGH.

B. Purification of CGH from CHO Culture Supernatant.

CGH was purified from CHO culture supernatant by established chromatographic procedures: first the CGH was captured on a strong cation exchanger, POROS HS50; next it was purified using Hydrophobic Interaction Chromatography with TosoHaas Butyl650S resin; and finally was polished and buffer-exchanged into PBS by Superdex 75 size exclusion chromatography.

C. Cation Exchange Chromatography.

The CHO culture supernatant was 0.2 μm filtered and adjusted to pH 6 and 20 mM 2-Morpholinoethanesulfonic Acid (MES). The CGH in the adjusted supernatant was captured at 55 cm/hr using a 1:2 online dilution with 20 mM MES pH 6 onto a POROS HS 50 column that was previously equilibrated in 20 mM MES pH 6. After loading was complete, the column was washed with 20 column volumes (CV) of equilibration buffer. This was followed by a 3 CV wash with 250 mM NaCl in 20 mM MES pH 6 at 90 cm/hr. Next the CGH was eluted from the column with 3 CV of 500 mM NaCl in 20 mM MES pH 6 at the same flow rate. Finally the column was stripped with steps of 1M and 2M NaCl and then re-equilibrated with 20 mM MES pH 6. The 500 mM NaCl-eluted pool containing the CGH was adjusted at room temperature to 1.0M with (NH₄)₂SO₄ and to pH 6.9 with NaOH for the next step.

D. Butyl 650S Hydrophobic Interaction Chromatography (HIC).

HIC is an adsorptive liquid chromatography technique that separates biomolecules on the basis of net hydrophobicity. The sample is bound to the gel in high salt and then a gradient or step elution of decreasing salt concentration is applied to elute the sample.

The adjusted pool of CGH from the cation exchange chromatography was applied directly at 100 cm/hr to the TosoHaas Butyl650S resin equilibrated in 50 mM NaH₂PO₄ pH 6.9 containing 1.0 M (NH₄)₂SO₄. After loading, the column was washed with 10 CV of equilibration buffer and 10 CV of 50 mM NaH₂PO₄ pH 6.9 containing 0.9M (NH₄)₂SO₄. The CGH was then eluted from the column at 200 cm/hr by reducing the (NH₄)₂SO₄ to 0.5M and collecting 5 CV. This CGH pool was concentrated via ultrafiltration using an Amicon stirred cell with a 5 kDa-cutoff membrane.

E. Size-Exclusion Chromatography.

The concentrated CGH pool was then applied to an appropriately sized bed of Superdex 75 resin (i.e. <5% of bed volume) for removal of remaining HMW contaminants and for buffer exchange into PBS. The CGH eluted from the Superdex 75 column at about 0.65 to 0.7 CV and was concentrated for storage at −80° C. using the Amicon stirred cell with a 5 kDa-cutoff ultrafiltration membrane. The heterodimeric protein was pure by Coomassie-stained SDS PAGE, had the correct NH2 termini, the correct amino acid composition, and the correct mass by SEC MALS. The overall process recovery estimated by RP HPLC assay was 50-60%.

Additionally, the CGH polypeptide can be expressed in other host systems. The production of recombinant polypeptides in cultured mammalian cells is disclosed by, for example, Levinson et al., U.S. Pat. No. 4,713,339; Hagen et al., U.S. Pat. No. 4,784,950; Palmiter et al, U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from. See, e.g., U.S. Pat. No. 4,956,288. Promoters include those from SV-40 or cytomegalovirus, metallothionein genes (U.S. Pat. Nos. 4,579,821 and 4,601,978) and the adenovirus major late promoter. Within an alternative embodiment, adenovirus vectors can be employed. See, for example, Garnier et al., Cytotechnol. 15:145-55, 1994.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

### EXAMPLE 6

### The CGH Receptor (TSH-R) is Expressed on Many Different Cells of the Peripheral Immune System

Whole blood (50 ml) was collected from a healthy human donor and mixed 1:1 with PBS in 50 ml conical tubes. Thirty ml of diluted blood was then underlayed with 15 ml of Ficoll Paque Plus (Pfizer-Pharmacia). These gradients were centrifuged 30 min at 500 g and allowed to stop without braking. The RBC-depleted cells at the interface (PBMC) were collected and washed 3 times with PBS.

Cells were resuspended in FACS Wash Buffer (WB=1×PBS/1% BSA/10 mM Hepes), counted in trypan blue, and 1×10⁶ viable cells of each type were aliquoted into wells of a 96-well round-bottomed plate. Cells were washed and pelleted, then incubated for 20 min on ice with 10 ug/ml of CGH-biotin and cocktails of fluorescently-labeled (FITC and CyChrome) monoclonal antibodies (PharMingen, San Diego, Calif.) recognizing various cell surface markers used to identify particular human immune cell subsets. These markers include the following (listed in the groups of 2 tested in combination with CGH-biotin or a media-only control): CD45RA/CD4, CD56/CD 16, CD45RA/CD8, CD 14/CD 16, CD3/CD 19. Cells were washed and then stained with 5 ug/ml streptavidin-PE (PharMingen) for an additional 20 min, to stain CGH-biotin-binding cells. Cells were washed thoroughly and pelleted, then resuspended in 0.4 ml of WB and analyzed on a FACSCalibur using CellQuest software (Becton Dickinson, Mountain View, Calif.).

As shown in TABLE 2, CGH-biotin clearly bound to monocytes, B cells, T cells (both CD4+ and CD8+, not shown), and to NK cells. Additionally, it appeared to bind more avidly to memory phenotype (CD45RA−) CD4+ T cells than to naïve (CD45RA+) CD4+ T cells. These data generally agree with the expression pattern of TSH-R determined by RT-PCR (see Example 3) and by immunoprecipitation studies (see Bagriacik and Klein, J. Immunol. 164: 6158-65, 2000).

TABLE 2
CGH-biotin binds to a wide variety of immune cells in human
peripheral blood.

| | | | FL-2 MFI: |
|---|---|---|---|
| | | FL-2 MFI: | CGH-biotin + |
| Cell Subset | Gated On: | 0 + SA-PE | SA-PE |
| Monocytes | CD14+ | 9.1 | 56.4 |
| B cells | CD19+ | 3.8 | 12.9 |
| T cells | CD3+ | 4.3 | 9.1 |
| NK cells | CD56+ | 3.6 | 11.9 |
| Naïve CD4+ T cells | CD4+ CD45RA+ | 3.5 | 4.9 |
| Memory CD4+ T cells | CD4+ CD45RA− | 3.4 | 8.7 |

Mean Fluorescence Intensities (MFI) are shown for CGH-biotin (followed by streptavidin-phycoerythrin [SA-PE]) staining of human PBMC, gated on various immune cell subsets. CGH-biotin was used at 10 ug/ml. SA-PE was purchased from Pharmingen and used at 5 μg/ml. These data are representative of 3 independent experiments with different blood donors.

### EXAMPLE 7

CGH Treatment Alters the Production of Inflammatory Cytokines in the LPS-Induced Mild Endotoxemia Mouse Model

An in vivo experiment was designed to examine the effect of CGH in a mouse model of LPS-induced mild endotoxemia. This model mimics acute endotoxemia/sepsis by challenging mice with a low, non-lethal dose of bacterial endotoxin (lipopolysaccharide, LPS). Serum is collected at various timepoints (1-8 hours) after intraperitoneal LPS injection and analyzed for altered expression of a wide variety of pro- and anti-inflammatory cytokines and acute phase proteins that mediate the inflammatory response. The model provides a means to assess the potential anti-inflammatory effects of therapeutic candidates during a robust inflammatory response. To initially assess the model, we measured proinflammatory cytokines in a pilot experiment to collect reference data for the model.

In this pilot study, six-month old Balb/c (Charles River Laboratories, Wilmington, Mass.) female mice were injected with 25 μg LPS (Sigma) in sterile PBS intraperitoneally (i.p.). Serum samples were collected at 0, 1, 4, 8, 16, 24, 48 and 72 hours from groups of 8 mice for each time point. Serum samples were assayed for inflammatory cytokine levels. IL-10, IL-6, TNFα, and IL-10 levels were measured using commercial ELISA kits purchased from Biosource International (Camarillo, Calif.).

TNFα levels peaked to 4000 pg/ml and IL-10 levels were 341 pg/ml at 1 hour post-LPS injection. At 4 hours post LPS injection, IL-6, IL-10 and IL-10 were 6,100 pg/ml, 299 pg/ml and 229 pg/ml, respectively. These results indicated that pro-inflammatory cytokines were indeed produced in this model. From the inflammatory mediators listed above, two were chosen as biological markers for the LPS model of mild endotoxemia: serum TNFα levels 1 hour post-LPS and serum IL-6 levels 4 hours post-LPS.

C57B1/6 mice (Charles River Laboratories; 5 mice/group) were treated i.p. with PBS, 0.2 mg/kg CGH in PBS, or 2 mg/kg CGH in PBS 1 hour prior to LPS challenge. The mice were then challenged with 25 ug of LPS i.p. and bled at 1 hour and 4 hours after LPS injection. Serum was analyzed for TNFα (1 hour) and IL-6 (4 hours) levels by ELISA.

Injection of 2 mg/kg CGH protein 1 hour prior to the LPS injection significantly reduced (by about 60%) the TNFα induction at the 1 hour time point, whereas CGH increased serum IL-6 levels by about 70% at the 4 hour time point (TABLE 3, Expt #1). Statistical significance was determined by an unpaired Student's t-test. Similar trends were observed using the lower dose of CGH (0.2 mg/kg), although the differences were not statistically significant (TABLE 3, p values). These results were consistently obtained in 3 independent experiments (TABLE 3). Thus, CGH can suppress the production of the pro-inflammatory cytokine TNFα, while enhancing expression of IL-6, a cytokine that can have either pro- or anti-inflammatory properties. This likely reflects the ability of CGH to increase cAMP levels in immune cells that express TSH-R, leading to changes in the synthesis and secretion of several inflammatory cytokines (see Example 3, Bagriacik and Klein, J Immunol 164: 6158-65, 2000, and Delgado and Ganea, J Biol Chem 274: 31930-40, 1999).

In another experiment, mice were treated with 2 mg/kg of either zlut1 or zsig51 and demonstrated that neither of these monomers had any effect on serum TNFα or IL-6 levels, indicating that the activity of CGH requires the complete heterodimer (data not shown). To determine if CGH would potentiate the effect of a sub-maximal dose of glucocorticoid, groups of 10 C57B1/6 mice each were treated i.p. with PBS, 0.15 or 1.5 mg/kg Dex, 2 mg/kg CGH, or a combination of CGH and low or high doses of Dex, 1 hour prior to injection of 25 ug LPS i.p. As shown in TABLE 3 (Expt #2), either 2 mg/kg CGH or 1.5 mg/kg Dex treatment alone (1 hour prior to LPS) caused a significant drop in serum TNFα levels at 1 hour, as observed in previous experiments. The effects of CGH administered in conjunction with Dex on inhibition of TNFα production were greater than either dose of Dex alone. In particular, the use of CGH with a low dose of Dex substantially decreased the elevation of serum TNFα compared to the low dose of Dex alone. As before, CGH treatment again enhanced increased serum IL-6 levels at 4 hours; however, serum IL-6 levels decreased significantly when the mice received either Dex alone (1.5 mg/kg) or a combination of CGH and Dex (TABLE 3). Thus, the serum IL-6 levels in Dex+CGH treated mice more closely resembled those in mice treated with Dex alone, rather than those treated with CGH alone, suggesting the activity of the glucocorticoid was dominant over that of CGH in this setting.

TABLE 3
CGH treatment reduces TNFα production, and increases IL-6
levels in the LPS-induced mild endotoxemia mouse model.

| | | | TNFα, | IL-6, | p-value | p-value |
|---|---|---|---|---|---|---|
| | TREATMENT | | pg/ml | ng/ml | vs. PBS | vs. PBS |
| EXPT # | (1 hr prior to LPS) | n = | 1 hour | 4 hours | TNFα | IL-6 |
| 1 | PBS | 5 | 5687 +/− 2310 | 41.1 +/− 10.0 | — | — |
| | 0.2 mg/kg CGH | 5 | 3916 +/− 1057 | 49.7 +/− 3.1 | 0.1576 | 0.1198 |
| | 2 mg/kg CGH | 5 | 2290 +/− 530 | 71.1 +/− 12.9 | 0.0125 | 0.0037 |
| 2 | PBS | 10 | 3115 +/− 891 | 41.2 +/− 11.9 | — | — |
| | 2 mg/kg CGH | 10 | 2274 +/− 524 | 57.1 +/− 16.2 | 0.0191 | 0.0224 |
| | 0.15 mg/kg Dex | 10 | 1765 +/− 589 | 37.1 +/− 8.8 | 0.0008 | 0.7883 |
| | 1.5 mg/kg Dex | 10 | 264 +/− 138 | 16.0 +/− 4.9 | 3.0 × 10⁻⁸ | 0.000008 |
| | 2 mg/kg CGH + | 10 | 955 +/− 349 | 40.7 +/− 14.8 | 0.000003.1 × 10⁻⁶ | 0.8607 |
| | 0.15 mg/kg Dex | | | | | |
| | 2 mg/kg CGH + 1.5 | 10 | 238 +/− 82 | 17.3 +/− 5.6 | 0.00000001 | 0.00002 |
| | mg/kg Dex | | | | | |

### EXAMPLE 8

### Delayed Type Hypersensitivity in CGH-Treated Mice

Delayed Type Hypersensitivity (DTH) is a measure of T cell responses to specific antigen. In this response, mice are immunized with a specific protein in adjuvant (e.g., chicken ovalbumin, OVA) and then later challenged with the same antigen (without adjuvant) in the ear. Increase in ear thickness (measured with calipers) after the challenge is a measure of specific immune response to the antigen. DTH is a form of cell-mediated immunity that occurs in three distinct phases 1) the cognitive phase, in which T cells recognize foreign protein antigens presented on the surface of antigen presenting cells (APCs), 2) the activation/sensitization phase, in which T cells secrete cytokines (especially interferon-gamma; IFN-γ) and proliferate, and 3) the effector phase, which includes both inflammation (including infiltration of activated macrophages and neutrophils) and the ultimate resolution of the infection. This reaction is the primary defense mechanism against intracellular bacteria, and can be induced by soluble protein antigens or chemically reactive haptens. A classical DTH response occurs in individuals challenged with purified protein derivative (PPD) from Myco bacterium tuberculosis (TB), when those individuals injected have recovered from primary TB or have been vaccinated against TB. Induration, the hallmark of DTH, is detectable by about 18 hours after injection of antigen and is maximal by 24-48 hours. The lag in the onset of palpable induration is the reason for naming the response “delayed type.” In all species, DTH reactions are critically dependent on the presence of antigen-sensitized CD4+ (and, to a lesser extent, CD8+) T cells, which produce the principal initiating cytokine involved in DTH, IFN-γ.

In order to test for anti-inflammatory effects of CGH, a DTH experiment was conducted with four groups of C57B1/6 mice treated with: I) PBS, II) 1.5 mg/kg Dexamethasone (Dex), III) 0.2 mg/kg CGH, and IV) 2 mg/kg CGH. All of these treatments were given intraperitoneally two hours prior to the OVA re-challenge. The mice (8 per group) were first immunized in the back with 100 ug chicken ovalbumin (OVA) emulsified in Ribi in a total volume of 200 ul. Seven days later, the mice were re-challenged intradermally in the left ear with 10 ul PBS (control) or in the right ear with 10 ug OVA in PBS (no adjuvant) in a volume of 10 ul. Ear thickness of all mice was measured before injecting mice in the ear (0 measurement). Ear thickness was measured 24 hours after challenge. The difference in ear thickness between the 0 measurement and the 24 hour measurement is shown in TABLE 4. Control mice in the PBS treatment group developed a strong DTH reaction as shown by increase in the ear thickness at 24 hours post-challenge (TABLE 4, Expt #1). In contrast, mice treated with Dex or CGH had a lesser degree of ear thickness compared to controls. These differences were statistically significant, as determined by Student's t-test (TABLE 4, p values vs. PBS).

TABLE 4
CGH inhibits the Delayed Type Hypersensitivity (DTH) reaction when
administered either at the challenge or at the sensitization phase of the response.

| | | | CHANGE IN EAR | | |
|---|---|---|---|---|---|
| | | | THICKNESS (×10⁻³ inch) | | |
| | | TIME/ROUTE OF | LEFT EAR | RIGHT EAR | p value vs. |
| EXPT # | TREATMENT | TREATMENT | (PBS) | (OVA) | PBS |
| | PBS | Challenge (d 7) | 0.64 +/− 0.88 | 5.89 +/− 2.32 | — |
| 1 | 1.5 mg/kg Dex | i.p. | 0.42 +/− 0.52 | 2.62 +/− 1.18 | 0.0020 |
| (n = 8) | 0.2 mg/kg CGH | | 0.17 +/− 0.95 | 3.48 +/− 0.79 | 0.0032 |
| | 2.0 mg/kg CGH | | 0.21 +/− 0.34 | 2.48 +/− 1.05 | 0.0145 |
| | PBS | Challenge (d 7) | 0.99 +/− 0.56 | 6.64 +/− 0.80 | — |
| 2 | 1.5 mg/kg Dex | i.p. | 0.23 +/− 0.77 | 2.89 +/− 1.29 | 0.000007 |
| (n = 8) | 0.2 mg/kg CGH | | 0.65 +/− 0.63 | 4.41 +/− 0.95 | 0.0002 |
| | 2.0 mg/kg CGH | | 0.67 +/− 1.05 | 3.92 +/− 1.00 | 0.00006 |
| | PBS | Sensitization | 1.50 +/− 0.53 | 7.78 +/− 1.70 | — |
| | | (d 0-4) | | | |
| 3 | 1.5 mg/kg Dex | i.p. | 0.50 +/− 0.54 | 4.38 +/− 1.34 | 0.0014 |
| (n = 7) | 0.2 mg/kg CGH | | 1.31 +/− 0.42 | 4.06 +/− 0.73 | 0.0004 |
| | 2.0 mg/kg CGH | | 1.11 +/− 0.49 | 4.57 +/− 1.58 | 0.0033 |

A second DTH experiment was performed to confirm these results (TABLE 4, Expt #2). Again, CGH and Dex-treated mice exhibited significantly reduced ear swelling in response to the OVA re-challenge (TABLE 4, Expt #2). In DTH experiment #3, CGH was evaluated for anti-inflammatory effects when administered during the sensitization phase of the reaction (i.e. when T cells are responding to the antigen). Mice (7 per group) were administered PBS, Dex or CGH intraperitoneally once a day from days 0 to 4. The mice were then re-challenged with OVA or PBS on day 7 and ear thickness was measured on day 8. Once again, both Dex and CGH significantly inhibited the DTH reaction (TABLE 4, Expt #3), suggesting that CGH can exert anti-inflammatory effects both early and late in the inflammation process.

Ears from mice in DTH experiment #1 were analyzed by immunohistochemistry to assess which cell types were most affected by CGH treatment. Ears were fixed in Zinc/Tris buffer (2.3 mM calcium acetate/31.6 mM zinc acetate/36.7 mM zinc chloride in 0.1M Tris-HCL buffer, pH 7.4) for 24 hours at room temperature and stained with antibodies specific for CD4, CD8, CD11c, and Gr-1 (neutrophils). Although we did not detect staining of CD4, CD8 or CD11c+cells, there were some interesting differences in the anti-Gr-1 stained sections. Ears were stained using a TechMate 500 autoimmunostainer (Biotech % Ventana) MIP protocol with some modifications. After drying the slides for 1 hour at 60° C., the sections were stained with a rat anti-mouse Gr-1 mAb (clone 7/4, Serotec, isotype rat IgG2a, used at 1.25 ug/ml final dilution), overnight at 4° C. This step was followed, after a wash, by biotinylated rabbit anti-rat IgG secondary antibody (Dako, used at 10 ug/ml in PBS with 2% normal rabbit serum and 2% nonfat dry milk) for 45 min. The sections were then washed and treated with HP Block (1.5% H₂O₂ in 50% methanol) 3 times, 7 min each, followed by 25 min in avidin-biotin complex, 3 times of 4 min each in DAB (Diaminobenzidine), then with Methyl green for 10 min.

From this staining procedure, there was a clear reduction in the number of neutrophils infiltrating the ears of those mice treated with CGH or Dex, compared to the PBS-treated controls. Histomorphometry was performed to obtain the average pixel density of neutrophils per unit length (1 mm) present in the ear samples. The results of these analyses are shown in TABLE 5. Despite a fair amount of variability among each group, there was a significant reduction in neutrophil staining in the Dex and low dose CGH groups, as well as a nearly significant (p=0.0507) reduction in the high dose CGH group (see TABLE 5, p values).

TABLE 5
CGH suppresses neutrophil infiltration in the ears of mice undergoing the DTH
response.
Average pixel density, from 4 fields per ear per mouse (n = 4/group).

| | | Pixel density: Neutrophils | | p value vs. | |
|---|---|---|---|---|---|
| | TIME OF | LEFT EAR | RIGHT EAR | PBS | |
| TREATMENT | TREATMENT | (PBS) | (OVA) | LEFT | RIGHT |
| PBS | Challenge | 293 +/− 378 | 12657 +/− 4431 | — | — |
| 1.5 mg/kg | (day 7) | 749 +/− 686 | 3651 +/− 2779 | 0.2877 | 0.0137 |
| Dex | i.p. | | | | |
| 0.2 mg/kg | | 544 +/− 1044 | 5605 +/− 3725 | 0.6661 | 0.0507 |
| CGH | | | | | |
| 2 mg/kg | | 273 +/− 399 | 5535 +/− 5331 | 0.9460 | 0.0856 |
| CGH | | | | | |

CGH anti-inflammatory effects do not seem to be mediated by an increase in corticosteroids by the adrenal cortex.

Since the receptor for CGH, TSH-R, is expressed by the adrenal glands, there was concern that the anti-inflammatory effects observed might be an indirect effect of increasing endogenous corticosteroid production in CGH-treated mice. One well-established side effect of increasing either exogenous or endogenous corticosteroid levels is substantial atrophy of the thymus, as the developing T cells are induced to undergo apoptosis. Therefore, the thymuses of the CGH and Dex-treated mice in the DTH experiments were analyzed. As shown in TABLE 6, while Dex-treated mice exhibited obvious thymic atrophy, neither the thymus weight nor the overall thymocyte cell counts were significantly affected by CGH treatment. Thymocytes were also analyzed by flow cytometry after staining the cells with fluorescently labeled antibodies to CD4, CD8 and CD3 (PharMingen, San Diego, Calif.), and it was found that the relative proportion of each thymocyte subset (CD4 single positive, CD8 single positive, CD4+ CD8+ double positive, and CD4−CD8− double negative) in CGH-treated mice was not significantly different from that of the PBS-treated group. Thus, CGH seems to be mediating its anti-inflammatory effects in a manner distinct from that of exgenous glucocorticoids like Dex. This should prove to be an important benefit, as many of the adverse side effects of glucocorticoid treatment might potentially be avoided with CGH therapy.

TABLE 6
Unlike glucocorticoid treatment, CGH treatment in vivo does not induce
thymic atrophy.
Thymuses were collected from mice in DTH expt #3 (in Table 5, above).

| | TIME OF | | | | |
|---|---|---|---|---|---|
| TREATMENT | TREATMENT | THYMUS | THYMOCYTE | | |
| GROUP | IN DTH | WEIGHT | COUNT | p value vs. PBS | |
| n = 7 | EXPT | (mg) | (×10⁻⁶ cells) | Weight | Counts |
| PBS | Sensitization | 61.9 +/− 12.4 | 140.3 +/− 36.6 | — | — |
| 1.5 mg/kg | (days 0-4) | 30.1 +/− 7.6 | 46.0 +/− 7.4 | 0.000004 | 0.0023 |
| Dex | i.p. | | | | |
| 0.2 mg/kg | | 60.6 +/− 4.8 | 134.0 +/− 24.0 | 0.8856 | 0.7848 |
| CGH | | | | | |
| 2.0 mg/kg | | 58.43 +/− 20.0 | 128.0 +/− 63.1 | 0.8001 | 0.7485 |
| CGH | | | | | |

### EXAMPLE 9

### Assessment of Lymphoid Tissues in Mice Treated Chronically with CGH

As described above, glucocorticoid treatment results in reductions in immune cell populations, resulting in increased risk of infection. In order to determine whether long-term treatment of mice with CGH might have a deleterious effect on the immune system, C57B1/6 mice were treated with either 300 ug/kg/day human CGH or with PBS (4 mice/group) for a total of 4 weeks. On the last day of treatment, the spleens, peripheral lymph nodes (pooled inguinal, cervical, axillary, and brachial nodes), and thymuses were collected from each group of mice, and single cell suspensions were prepared. Spleens were crushed between two frosted glass slides, while thymuses and lymph nodes were teased apart with forceps, and the cells released were passed over a Nytex membrane (cell strainer) and pelleted. Cells were resuspended in FACS wash buffer (WB=1× Hank's balanced salt solution/1% BSA/10 mM hepes), counted in trypan blue, and 1×10⁶ viable cells of each type were aliquoted into wells of a 96-well round bottom plate. Cells were washed and pelleted, then incubated for 20 min on ice with cocktails of fluorescently-labeled (FITC, PE, and CyChrome) monoclonal antibodies (PharMingen, San Diego, Calif.) recognizing various cell surface markers used to identify particular immune cell subsets. These markers include the following (listed in the groups of 3 tested in combination). For spleen staining: CD11b/Gr1/B220, CD4/CD44/CD8, DX5/NK1.1/CD3; for lymph node staining: CD62L/CD44/CD4, CD62L/CD44/CD8, and CD11b/Gr1/B220; and for thymus staining: CD4/CD3/CD8. Cells were washed thoroughly and pelleted, then resuspended in 0.4 ml of WB and analyzed on a FACSCalibur using CellQuest software (Becton Dickinson, Mountain View, Calif.). As shown in TABLE 7, there were no significant differences (as determined by Student's t-test) in the number (or percentage; data not shown) of each cell population in the lymphoid tissues from the PBS vs. CGH-treated groups of mice.

TABLE 7
Chronic CGH treatment of normal mice treated with 300 ug/kg/day of CGH for
4 weeks does not affect the cellular distribution in their lymphoid tissues.

| | | NUMBER OF CELLS IN EACH | | |
|---|---|---|---|---|
| | | SUBPOPULATION (millions) | | |
| | | AVERAGE +/− STD | | |
| | | DEVIATION | | |
| | | PBS- | CGH- | |
| | GATED CELL | TREATED | TREATED | |
| TISSUE | POPULATION | GROUP | GROUP | p value |
| Spleen | TOTAL | 91.8 +/− 21.5 | 99.0 +/− 20.6 | 0.6464 |
| | CD4+ (T cells) | 18.8 +/− 2.60 | 15.3 +/− 3.39 | 0.1475 |
| | CD8+ (T cells) | 12.8 +/− 2.18 | 10.9 +/− 2.47 | 0.3095 |
| | B220+ (B cells) | 54.9 +/− 16.2 | 59.1 +/− 13.28 | 0.7046 |
| | CD11b+Gr1-low | 0.97 +/− 0.43 | 3.40 +/− 3.43 | 0.2091 |
| | (monocytes) | | | |
| | CD11b+Gr1-high | 2.69 +/− 1.37 | 7.35 +/− 7.41 | 0.2624 |
| | (activated | | | |
| | granulocytes) | | | |
| | CD11b−Gr1+ | 5.10 +/− 1.17 | 6.57 +/− 1.21 | 0.1323 |
| | (granulocytes) | | | |
| | NK1.1+DX5+ (NK) | 2.32 +/− 0.69 | 3.68 +/− 1.17 | 0.0912 |
| Peripheral | TOTAL | 11.3 +/− 0.60 | 10.9 +/− 4.05 | 0.8552 |
| Lymph | CD4+ | 3.32 +/− 0.44 | 3.23 +/− 1.71 | 0.9290 |
| Nodes | CD8+ | 2.33 +/− 0.29 | 2.54 +/− 1.65 | 0.8124 |
| | B220+ | 6.50 +/− 0.43 | 6.65 +/− 2.54 | 0.9067 |
| Thymus | TOTAL | 111.0 +/− 18.4 | 93.6 +/− 10.0 | 0.1486 |
| | CD4+CD8+ (DP) | 95.2 +/− 15.7 | 79.9 +/− 8.92 | 0.1443 |
| | CD4−CD8− (DN) | 4.36 +/− 0.77 | 4.06 +/− 0.51 | 0.5350 |
| | CD4+CD8− (CD4 SP) | 7.28 +/− 1.28 | 5.72 +/− 0.45 | 0.0616 |
| | CD4− CD8+ (CD8 SP) | 3.91 +/− 0.75 | 3.65 +/− 0.56 | 0.5864 |

These results suggest that although CGH has potent anti-inflammatory activity in vivo, treatment with CGH does not result in depletion of important immune cell populations in the cellular compartments investigated.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for treating inflammation, comprising administering a therapeutically sufficient amount of a CGH polypeptide to a mammal, wherein administration of the polypeptide results in a clinically significant improvement in the inflammatory condition of the mammal.

2. The method according to claim 1, wherein the CGH polypeptide forms a heterodimer, comprising the amino acid sequence as shown in SEQ ID NO:3, and the amino acid sequence as shown in SEQ ID NO:6.

3. The method according to claim 2, wherein the clinically significant improvement in the inflammatory condition is selected from the group consisting of:
a) a decrease or inhibition in pain;
b) a decrease or inhibition in swelling;
c) a decrease or inhibition in redness;
d) a decrease or inhibition in heat;
e) and a decrease or inhibition in loss of function.

4. The method according to claim 2, wherein the inflammation is acute or chronic.

5. The method according to claim 2, wherein the inflammation or inflammatory condition is associated with an autoimmune disease.

6. The method according to claim 2, wherein the improvement is a reduction in inflammation.

7. The method according to claim 6, wherein the inflammation is acute or chronic.

8. The method according to claim 7, wherein the inflammation or inflammatory condition is associated with an autoimmune disease.

9. A method for treating inflammation, comprising administering a therapeutically sufficient amount of a CGH polypeptide to a mammal in conjunction with one or more glucocorticoids, wherein administration of the polypeptide results in a clinically significant improvement in the inflammatory condition of the mammal.

10. The method according to claim 9, wherein the CGH polypeptide forms a heterodimer, comprising the amino acid sequence as shown in SEQ ID NO:3, and the amino acid sequence as shown in SEQ ID NO:6.

11. The method according to claim 9, wherein the clinically significant improvement in the inflammatory condition is selected from the group consisting of:
a) a decrease or inhibition in pain;
b) a decrease or inhibition in swelling;
c) a decrease or inhibition in redness;
d) a decrease or inhibition in heat; and
e) a decrease or inhibition in loss of function.

12. The method according to claim 9, wherein administration of the polypeptide prevents or reduces a glucocorticoid-induced adverse side-effect.

13. The method according to claim 12, wherein the glucocorticoid-induced adverse side-effect is selected from the group consisting of: adrenocortical suppression, osteoporosis, bone necrosis, steroid-induced cataracts, steroid-induced obesity, corticosteroid-induced psychosis, gastrointestinal hemorrhage, thymic atrophy, and benign intracranial hypertension.

14. The method according to claim 9, wherein inflammation is acute or chronic.

15. The method according to claim 14, wherein inflammation or inflammatory condition is associated with an autoimmune disease.

16. A method for reducing inflammation, comprising administering a therapeutically sufficient amount of a CGH polypeptide to a mammal in conjunction with one or more glucocorticoids, wherein administration of the polypeptide results in a clinically significant improvement in the inflammatory condition of the mammal.

17. The method according to claim 16, wherein the CGH polypeptide forms a heterodimer, comprising the amino acid sequence as shown in SEQ ID NO:3, and the amino acid sequence as shown in SEQ ID NO:6.

18. The method according to claim 17, wherein the clinically significant improvement in the inflammatory condition is selected from the group consisting of:
a) a decrease or inhibition in pain;
b) a decrease or inhibition in swelling;
c) a decrease or inhibition in redness;
d) a decrease or inhibition in heat; and
e) a decrease or inhibition in loss of function.

19. The method according to claim 18, wherein the CGH polypeptide and the glucocorticoid are administered concurrently.

20. The method according to claim 18, wherein the CGH polypeptide and the glucocorticoid are administered sequentially.
